SEQUENCE LISTING (6 sequences)

SEQ ID NO: 1 - protein, 6 aa, synthetic construct
GFOGER

SEQ ID NO: 2 - protein, 6 aa, synthetic construct
GROGER

SEQ ID NO: 3 - protein, 6 aa, synthetic construct
GLOGEN

SEQ ID NO: 4 - protein, 6 aa, synthetic construct
GMOGER

SEQ ID NO: 5 - protein, 29 aa, synthetic construct
GXGPOGPOGPOGPOGPOGPOGPOGPOGXG

SEQ ID NO: 6 - protein, 36 aa, unidentified
GXGGPOGPOGPOGPOGFOGERGPOGPOGPOGPOGXG

(19) United States
(12) Patent Application Publication
(10) Pub. No.: US 2026/0241071 A1
(43) Pub. Date: Aug. 20, 2026

(54) PEPTIDES AND HYDROGELS FOR TREATING CORNEAL DISEASES

(71) Applicants: University of Ottawa, Ottawa (CA); Ottawa Heart Institute Research Corporation, Ottawa (CA); RSEM, Limited Partnership, Québec City (CA)

(72) Inventors: Emilio Isaac ALARCON ABARZUA, Russell (CA); Marcelo Alejandro MUNZO FIGUEROA, Ottawa (CA); Aiden James VILÉ MACADAM, Ottawa (CA); Alex ROSS, Ottawa (CA); Michel GRENIER, Ottawa (CA); May GRIFFITH, Montreal (CA); Isabelle BRUNETTE, Verdun (CA)

(21) Appl. No.: 19/162,651

(22) PCT Filed: Mar. 6, 2024
(86) PCT No.: PCT/CA2024/050276

§ 371 Date: Sep. 5, 2025

Related U.S. Application Data
(60) Provisional application No. 63/450,139, filed on Mar. 6, 2023.

(51) Int. Cl. A61L 27/24; A61L 27/18; A61L 27/20; A61L 27/26; A61L 27/50; A61L 27/52; A61L 27/54

(52) U.S. Cl. CPC A61L 27/24; A61L 27/18; A61L 27/20; A61L 27/26; A61L 27/50; A61L 27/52; A61L 27/54

(57) ABSTRACT

Provided herein are photosensitive bulking agent (PBA) comprising a photoreactive collagen-like peptide (PCLP) and polyethylene glycol (PEG). Also provided herein are compositions, scaffolds, precursor hydrogels and hydrogels comprising the PBA and methods of preparation thereof. Photoreactive collagen-like peptides comprising photoreactive residues are also provided herein. Compositions comprising photoreactive collagen-like peptides that are not crosslinked, partially crosslinked and fully crosslinked are provided as well as uses thereof for treating, preventing, or augmenting corneal thinning or any other corneal defects in a subject in need thereof. A method of treating a condition of the eye characterized by a corneal defect is also described herein. The method comprises making a limbal incision in an area affected by the corneal defect in a subject, administering a precursor hydrogel over the corneal defect, and irradiating the precursor hydrogel with visible light.

### FIELD OF INVENTION

The present invention relates generally to peptides, hydrogels, and methods of preparing hydrogels. More specifically, the present invention relates to hydrogels and methods of preparing hydrogels for treating corneal diseases.

### BACKGROUND

Historically, naturally occurring polymers have been used as building blocks in the synthesis of materials with therapeutic potential for organ and tissue repair. While some of those materials are already in clinical use, the room for precise chemical modification and functionalization plus batch-to-batch variability remain drawbacks for further expanding their therapeutic application. Further, the concept of “one size to fit all needs” is unrealistic when considering, for example, the intrinsic differences in physical properties amongst organs and tissues in the human body.

In recent years, the use of synthetic constructs, whose properties can be finely tuned, has gained considerable interest, with some successful examples for soft organ repair (2). Amongst the many synthetic materials, short peptide-based materials (<50 amino acids long) present several advantages, which includes relatively low cost of manufacturing at clinical grade and flexibility for further engineering (i.e. chemical modifications). However, the use of external chemical crosslinkers for assembling 3D matrices is detrimental for their use in applications such as stem cell delivery and has largely limited their application in the form of pre-crosslinked implants (3). To further add to this, precise design of peptide sequences must encompass self-assembly of the chains in the crosslinked state to strengthen the resulting mechanical properties of the material. Some examples in the literature report bio-orthogonal assembly of peptide-based hydrogels, mainly using cysteine containing peptides (4) and over the years there have been multiple papers using this style of chemistry. However, combination of this chemistry with collagen-like peptides (CLPs) remains for the most part underdeveloped (5). CLPs mimic the tertiary structure of collagen, a common protein that provides structural strength to tissues, and are often composed of Glycine-Proline-Proline (GPP) or Glycine-Proline-Hydroxyproline (GPO) triplets with a minimal sequence requirement of GXY (6). Their self-assembly characteristics can contribute to network crosslinking as three monomers combine into a trimer.

The cornea is the transparent outer eye structure directing light internally for vision and is responsible for two-thirds of the eye's focusing power (1). Vision loss is responsible for an estimated US$410 billion globally due to annual productivity losses and reduced quality of life for affected individuals (2). Corneal diseases resulting in permanent loss of transparency are among the most common causes of blindness, affecting 57 million worldwide, and are rising in prevalence as the population increases and ages (3). However, conditions where optical clarity is maintained but vision is distorted due to corneal thinning are also problematic. Corneal thinning disorders can be associated with degenerative, autoimmune, or infectious causes and mechanical trauma from contact lens wear. These non-inflammatory disorders are often associated with progressive corneal thinning and steepening or a bulging resulting in distortion and substantial vision (4, 5).

Keratoconus is an example of a common corneal thinning disease that affects 21 per 1000 men and 18 per 1000 women (5). While the etiology beyond both genetic and environmental origins is unclear, inflammation and oxidative stress have roles in disease development and progression (6). The hallmarks of keratoconus are excessive degradation of the collagen fibers by matrix metalloproteases and loss of corneal stromal keratocytes (7). Currently, the early stages of keratoconus are managed with therapeutic contact lenses, while later stages are treated by corneal crosslinking to stabilize remaining collagen and prevent further degradation. However, in severe cases, corneal transplantation (keratoplasty) is needed (8).

In corneal crosslinking, the collagen fibrils of the thinned stroma are typically crosslinked by UV light in the presence of riboflavin, which acts as a photosensitizer (9). When exposed to UVA light, riboflavin generates reactive oxygen species that induce the formation of covalent bonds both between collagen molecules and between collagen molecules and proteoglycans (10). The normal human cornea is about 550 μm thick centrally. For very thin corneas (between 350-400 μm), hypo-osmolar riboflavin is used to thicken the cornea to a minimum of 400 μm, to protect the endothelium that is UV sensitive (11).

Although corneal transplantation for severe keratoconus, where collagen crosslinking cannot be performed, is highly successful, complications include severe postoperative astigmatism, delayed visual rehabilitation, and graft rejection. Furthermore, a lack of donor corneas, particularly in developing countries, results in less than 5% of individuals in need receiving a corneal transplant (12). Even though biomaterials-based alternatives such as our solid corneal implants made from recombinant human collagen that stimulated in situ regeneration of corneal tissue and as an alternative to donor human corneal transplantation (13, 14), they require invasive surgery. More recently, we showed that synthetic collagen analogs comprising short, collagen-like peptides (CLPs) conjugated to eight-arm polyethylene glycol (PEG) could be developed as a liquid filler (15). The CLP-PEG matrix incorporating fibrin glue was able to replace corneal tissue in an anterior lamellar keratoplasty procedure where 70% of the outer epithelium and stroma was removed (11).

Here, we expanded on the concept of using an injectable peptide-based material, but as a bulking agent to rebuild cornea stromas with advanced thinning in place of simply swelling the cornea with water to allow UV crosslinking (which risks endothelial damage) or the need for invasive donor cornea transplantation. As these thinned corneas have an abnormal extracellular matrix, we will substitute not just the collagen, but the corneal extracellular matrix which contains a significant proportion of water-retaining proteoglycans, to ensure optimal hydration of the rebuilt corneal stroma (16).

Photopolymerization using longer wavelengths (i.e., visible light) is typically safer than UV light and thus has become a promising research area for cornea biomaterials. Sani and colleagues formulated a gelatin-based bioadhesive, GelCORE, that could be crosslinked in situ using visible light (450 nm to 550 nm) to form a transparent hydrogel that could seal perforations and promote regeneration (17). The bioengineered hydrogels promoted cornea re-epithelization and displayed similar mechanical properties to the native cornea. However, this 2-week study in rabbits did not provide sufficient time to investigate the longer effects of this material. Further, the GELCORE formulation used methacrylated gelatin (GelMA), and gelatin of animal origin could carry risks such as allergy or zoonotic pathogen transmission (18). In addition, this formulation included triethanolamine, a chemical reported to cause eye irritation and liver tumours in animal studies (19). Further, the high UV irradiance used for GELCORE activation (100 mW/cm2) is sufficient to damage human retina pigment epithelium and would likely damage corneal endothelial cells in thin corneas (20, 21).

Alternative, additional, and/or improved hydrogels, and methods for the production thereof, are desirable.

### SUMMARY OF INVENTION

Provided herein are peptides, scaffolds, hydrogels, precursor hydrogels thereof, compositions thereof and uses thereof. Photosensitive bulking agents (PBA) comprising peptides, scaffolds, hydrogels, and/or precursor hydrogels are provided herein. Also provided herein are methods of preparing hydrogels and methods of treating corneal diseases.

In an embodiment, there is provided a photosensitive bulking agent (PBA) comprising a photoreactive collagen-like peptide (PCLP) and polyethylene glycol (PEG).

In an embodiment, the PBA comprises riboflavin.

In an embodiment, the PEG is a 2-Arms-PEG acrylate, 4-Arms-PEG acrylate or an 8-Arms-PEG acrylate, preferably an 8-Arms-PEG acrylate.

In an embodiment, the PCLP comprises at least one photoreactive residue.

In an embodiment, each of the at least one photoreactive residue is flanked by two aliphatic amino acid residues selected from the group consisting of glycine residues, alanine residues, valine residues and a combination thereof. Preferably, the two aliphatic amino acid residues are glycine residues.

In an embodiment, the PCLP comprises a plurality of collagen-like folding motifs.

In an embodiment, the PCLP comprises at least one cell adhesion motif flanked by the plurality of collagen-like folding motifs.

In an embodiment, the at least one cell adhesion motif comprises the amino acid residues GFOGER (SEQ ID NO: 1).

In an embodiment, the PBA is sensitive to visible light such as visible light having a wavelength of about 400 nm to about 490 nm.

In an embodiment, the visible light is blue light such as blue light having a wavelength of about 460 nm.

In an embodiment, the PCLP comprises the amino acid sequence defined by (X₀)ₘX₁(X₆)ₙ(X₂)ₒ(X₇)ₚX₅(X₈)_{q}; wherein each of X₀, X₆, X₇, and X₈ independently represent a glycine, an alanine or a valine, wherein 1≤m≤6, 1≤n≤6, 1≤q≤6 and 0≤p≤6; X₁ and X₅ are photoreactive residues which are the same or different; and X₂ represent collagen-like folding motifs, wherein 3≤o≤20.

In an embodiment, each of X₁ and X₅ represent a lysine, cysteine, tryptophan, tyrosine, or histidine protected by a N-allyloxycarbonyl (Alloc) group, a 9-fluorenylmethoxycarbonyl (Fmoc) group or a tert-butyloxycarbonyl (Boc) group, preferably each of X₁ and X₅ represent a lysine protected by a N-allyloxycarbonyl (Alloc) group.

In an embodiment, X₂ is defined by amino acid sequence GPO, POG, RGD, GROGER (SEQ ID NO:2), GLOGEN (SEQ ID NO:3), or GMOGER (SEQ ID NO:4), preferably POG.

In an embodiment, o=4.

In an embodiment, each of X₀, X₆, X₇, and X₈ represent a glycine.

In an embodiment, each of m, n, q=1 and p=0.

In an embodiment, the PCLP comprises the amino acid sequence defined by GK(Alloc)GPOGPOGPOGPOGPOGPOGPOGPOGK(Alloc)G (SEQ ID NO:5), wherein amino acid residue K(Alloc) represents a lysine protected by a N-allyloxycarbonyl (Alloc) group.

In an embodiment, the photoreactive collagen-like peptide (PCLP) comprises the amino acid sequence defined by (X₀)ₘX₁(X₆)ₙ(X₂)ₒX₃(X₄)ₚ(X₇)_{g}X₅(X₈)ᵣ; wherein each of X₀, X₆, X₇, and X₈ independently represent a glycine, an alanine or a valine, wherein 1≤m≤6, 1≤n≤6, 1≤q≤6 and 1≤r≤6; X₁ and X₅ are photoreactive residues which are the same or different; X₂ and X₄ are collagen-like folding motifs, wherein X₂ and X₄ are the same or different and 2≤o≤10 and 2≤p≤10; and X₃ represent at least one cell adhesion motif.

In an embodiment, each of X₁ and X₅ represent a lysine, cysteine, tryptophan, tyrosine, or histidine protected by a N-allyloxycarbonyl (Alloc) group, a 9-fluorenylmethoxycarbonyl (Fmoc) group or a tert-butyloxycarbonyl (Boc) group, preferably each of X₁ and X₅ represent a lysine protected by a N-Allyloxycarbonyl (Alloc) group.

In an embodiment, each of X₂ and X₄ is defined by amino acid sequence GPO, POG, RGD, GROGER (SEQ ID NO:2), GLOGEN (SEQ ID NO:3), or GMOGER (SEQ ID NO:4), preferably each of X₂ and X₄ is defined by amino acid sequence GPO.

In an embodiment, each of o and p=4.

In an embodiment, each of X₀, X₆, X₇, and X₈ represents a glycine.

In an embodiment, each of m, n, q, and r=1.

In an embodiment, the PCLP comprises the amino acid sequence defined by GK(Alloc)GGPOGPOGPOGPOGFOGERGPOGPOGPOGPOGK(Alloc)G (SEQ ID NO:6), wherein amino acid residue K(Alloc) represents a lysine protected by a N-allyloxycarbonyl (Alloc) group.

In an embodiment, the PBA is a photosensitive corneal stromal bulking agent.

In an embodiment, there is provided a composition comprising the PBA as described herein and a physiologically acceptable carrier.

In an embodiment, there is provided a scaffold comprising the PBA as described herein.

In an embodiment, the scaffold further comprises hyaluronic acid methacrylate and/or chondroitin methacrylate.

In an embodiment, there is provided a precursor hydrogel obtained by crosslinking a precursor solution comprising the PBA as described herein.

In an embodiment, the precursor hydrogel further comprises hyaluronic acid methacrylate and/or chondroitin methacrylate.

In an embodiment, the precursor hydrogel is obtained by crosslinking the precursor solution by irradiating the precursor solution with visible light in vitro.

In an embodiment, the visible light has a wavelength of about 400 nm to about 490 nm.

In an embodiment, the visible light is blue light such as blue light having a wavelength of about 460 nm.

In an embodiment, the precursor hydrogel has a viscosity within about 10% of the viscosity of Viscoat®.

In an embodiment, the precursor hydrogel is for use in preparing a hydrogel.

In an embodiment, the precursor hydrogel is for use in intrastromal injection.

In an embodiment, the precursor hydrogel is for use in treating a corneal disease.

In an embodiment, the precursor hydrogel is for use in treating a corneal disease characterized by a thinning cornea.

In an embodiment, the precursor hydrogel is for use in combination with a cornea implant for treating a corneal disease.

In an embodiment, there is provided a hydrogel comprising the precursor gel as described herein, wherein the hydrogel is obtained by crosslinking the precursor gel in vivo.

In an embodiment, crosslinking the precursor gel in vivo is performed by irradiating a subject injected with the precursor solution.

In an embodiment, the hydrogel is obtained by irradiating the precursor gel with visible light such as visible light has a wavelength of about 400 nm to about 490 nm.

In an embodiment, the visible light is blue light such as blue light having a wavelength of about 460 nm.

In an embodiment, the hydrogel comprises pores having a diameter of between about 10 μm to about 50 μm.

In an embodiment, the hydrogel has a compression modulus within about 10% of the compression modulus of a mammalian cornea.

In an embodiment, the hydrogel has a shear rate of between about 0 s⁻¹ to about 200 s⁻¹.

In an embodiment, the crosslinking allows conjugation of the photoreactive collagen-like peptide (PCLP) with the polyethylene glycol (PEG).

In an embodiment, there is provided a method of preparing a hydrogel in vivo comprising: providing a precursor solution comprising a photoreactive collagen-like peptide (PCLP), polyethylene glycol (PEG), and riboflavin; precrosslinking the precursor solution by irradiating the precursor solution with visible light to obtain a precursor hydrogel; injecting the precursor gel at a desired location in a subject; and crosslinking the precursor gel by irradiating the desired location in the subject with visible light to obtain a hydrogel in the subject.

In an embodiment, the photoreactive collagen-like peptide (PCLP) comprises at least one photoreactive residue.

In an embodiment, each of the at least one photoreactive residues is flanked by two glycine residues.

In an embodiment, the photoreactive collagen-like peptide (PCLP) comprises a plurality of collagen-like folding residues.

In an embodiment, the photoreactive collagen-like peptide (PCLP) comprises at least one cell adhesion motif flanked by the plurality of collagen-like folding residues.

In an embodiment, the precursor solution comprises hyaluronic acid methacrylate, gelatin methacrylate, chondroitin methacrylate or any combination thereof.

In an embodiment, the visible light has a wavelength of about 400 nm to about 490 nm.

In an embodiment, the visible light is blue light such as blue light comprising a wavelength of about 460 nm.

In an embodiment, the precrosslinking and/or crosslinking are performed under low oxygen concentration.

In an embodiment, the low oxygen concentration is about 1% v/v.

In an embodiment, precrosslinking the precursor solution is performed at 114 mW/cm².

In an embodiment, the precrosslinked precursor solution is centrifuged to remove bubbles therein.

In an embodiment, irradiating the precursor solution and/or irradiating the desired location in the subject is performed by pulsed irradiation.

In an embodiment, the pulsed irradiation comprises irradiating for about 0.5 s to about 5 seconds at intervals of about 0.5 s to about 5 seconds.

In an embodiment, a total light dosage irradiated at the desired location in the subject is about 2.5 J/cm².

In an embodiment, the PCLP comprises at least one cell adhesion motif.

In an embodiment, the hydrogel has a shear rate of between about 0 s⁻¹ to about 200 s⁻¹.

In an embodiment, the photoreactive collagen-like peptide (PCLP) comprises the amino acid sequence defined by (X₀)ₘX₁(X₆)ₙ(X₂)ₒX₃(X₄)ₚ(X₇)_{g}X₅(X₈)ᵣ; wherein each of X₀, X₆, X₇, and X₈ independently represents a glycine, an alanine, or a valine, wherein 1≤m≤6, 1≤n≤6, 1≤q≤6 and 1≤r≤6; X₁ and X₅ are photoreactive residues which are the same or different; X₂ and X₄ are collagen-like folding motifs, wherein X₂ and X₄ are the same or different and 2≤o≤10 and 2≤p≤10; and X₃ represent at least one cell adhesion motif.

In an embodiment, each of X₁ and X₅ represent a lysine, cysteine, tryptophan, tyrosine, or histidine protected by a N-allyloxycarbonyl (Alloc) group, a 9-fluorenylmethoxycarbonyl (Fmoc) group or a tert-butyloxycarbonyl (Boc) group, preferably each of X₁ and X₅ represent a lysine protected by a N-allyloxycarbonyl (Alloc) group.

In an embodiment, each of X₂ and X₄ is defined by amino acid sequence GPO, POG, RGD, GROGER (SEQ ID NO:2), GLOGEN (SEQ ID NO:3), or GMOGER (SEQ ID NO:4), preferably GPO.

In an embodiment, each of o and p=4

In an embodiment, each of X₀, X₆, X₇, and X₈ represents a glycine.

In an embodiment, each of m, n, q, and r=1.

In an embodiment, the PCLP comprises the amino acid sequence defined by GK(Alloc)GGPOGPOGPOGPOGFOGERGPOGPOGPOGPOGK(Alloc)G (SEQ ID NO:6), wherein amino acid residue K(Alloc) represents a lysine protected by a N-allyloxycarbonyl (Alloc) group.

In an embodiment, the hydrogel has a viscosity within about 10% of the viscosity of Viscoat®.

In an embodiment, the hydrogel has compression modulus within about 10% of the compression modulus of a mammalian cornea.

In an embodiment, crosslinking the precursor gel is performed by irradiating the desired location in the subject with visible light for a period of between about 2 minutes to about 15 minutes.

In an embodiment, there is provided a cornea implant comprising the precursor hydrogel as described herein or the hydrogel as described herein.

In an embodiment, there is provided a method of treating a condition of the eye characterized by a corneal defect, said method comprising: making a limbal incision in an area affected by the corneal defect in a subject; administering the precursor hydrogel over the corneal defect; and irradiating the precursor hydrogel with visible light.

In an embodiment, there is provided a peptide comprising the amino acid sequence GK(Alloc)GPOGPOGPOGPOGPOGPOGPOGPOGK(Alloc)G (SEQ ID NO:5), wherein amino acid residue K(Alloc) represents a lysine protected by a N-allyloxycarbonyl (Alloc) group.

In an embodiment, there is provided a peptide comprising the amino acid sequence GK(Alloc)GGPOGPOGPOGPOGFOGERGPOGPOGPOGPOGK(Alloc)G (SEQ ID NO:6), wherein amino acid residue K(Alloc) represents a lysine protected by a N-allyloxycarbonyl (Alloc) group.

In an embodiment, there is provided a composition comprising the peptide as described herein and an 8-Arms-PEG acrylate.

In an embodiment, the composition is not crosslinked.

In an embodiment, the composition is partially crosslinked.

In an embodiment, the composition is fully crosslinked.

In an embodiment, there is provided a use of the composition as described herein for treating, preventing, or augmenting corneal thinning or any other corneal defects in a subject in need thereof.

Other and further aspects and advantages of the present invention will be better understood upon the reading of the illustrative embodiments about to be described or will be indicated in the appended claims, and various advantages not referred to herein will occur to one skilled in the art upon employment of the invention in practice.

### BRIEF DESCRIPTION OF DRAWINGS

These and other features will become better understood with regard to the following description and accompanying drawings, wherein:

FIG. 1 shows steps 1 and 2 of the development of a low dosage blue-light activated material for in situ corneal repair using collagen-like peptides (CLPs) to replace gelatin, the initial test polymer. Step 3 depicts the creation of a library for the different compositions of the formulations. Pulses of blue light were applied steps 2, 6, and 7. The bottom shows the custom-designed peptides with photoreactive moieties identified as peptide 1 and peptide 2, according to an embodiment;

FIG. 2 shows a custom-made humidity chamber and blue light irradiation system used to mimic the cornea extracellular environment, according to an embodiment.

FIG. 3 shows that the light pulsation rate used during hydrogel formation impacts the degree of hydrogel swelling, according to an embodiment.

FIG. 4 shows that blue light pulsation does not affect cornea structure in a feline model: (Left) Representative in vivo confocal images for different cornea layers (epithelium, stroma, endothelium) measured before and after blue light irradiation. (Right) Representative 3D corneal tomography maps for feline eyes prior and after blue light irradiation. No significant difference was observed between the pre- and post-exposure maps. (Bottom) Changes in cornea thickness before and after blue light irradiation. Corneal thickness within the norms of species (0.578±0.064 mm) throughout the follow-up period.

FIG. 5 shows the identification of the optimal composition for a peptide-based light-activated cornea filler using methacrylated gelatin (GelMa): (A) Schematic depiction for the main steps used in the preparation of the light activated materials. Left: Precrosslinking step to increase viscosity produce materials that can withstand the high intracorneal pressure immediately post-injection and can be rapidly crosslinked using blue light. Middle: Precrosslinked formulation dispensed in a 96-well plate to be irradiated under low oxygen concentration and high humidity. Right: Irradiation of the samples with a fiber optic blue light source (total energy dose of 2.5 J/cm²). (B) Total light dosage needed to precrosslink (PXL) the formulations composed of 8-Arms-PEG acrylate, chondroitin methacrylate (MC), hyaluronic methacrylate (HC), and gelatin methacrylate (GM), concentrations expressed in % w/v. (C) Total light dosage needed to precrosslink (PXL) the formulations composed of 8-Arms-PEG acrylate, chondroitin methacrylate (MC), hyaluronic methacrylate (HC), and gelatin methacrylate (GM) with and without APS/TEMED. (D) Total light dosage needed to solidify the formulations composed of 8-Arms-PEG acrylate, chondroitin methacrylate (MC), hyaluronic methacrylate (HC), and our custom-designed peptides (see main text for further details). (E) Light transmittance (380 nm-750 nm) for the formulations prepared using our custom-made peptide formulations. (F) Water content of the top four hydrogel candidates swollen in dextran solution for 1 day or 2 weeks and synthesized from either freshly prepared injectable formulations or formulations stored at 4° C. for 5 weeks shielded from light.

FIG. 6 shows the physical characterization of hydrogels with varying concentrations of HM, CM, and GM. Hydrogels were composed of 8-arm-PEG acrylate and varying concentrations of gelatin methacrylate (GM), hyaluronic acid methacrylate (HM), and chondroitin methacrylate (CM) (A) The average transmittance of hydrogels for the entire visible spectrum. The average transmittance was taken as the average of the maximum (750 nm) and minimum transmittance (350 nm). (B) Percent swelling of hydrogels in PBS for 24 h at 37° C. (C) Refractive index of hydrogels measured using an Abbemat refractometer at 37° C. (D) Shear thinning index of hydrogels before final crosslinking step. Shear thinning index was taken as the viscosity at low shear rate (0-3 s⁻¹) over the viscosity at high shear rate (200 s⁻¹).

FIG. 7 shows the energy dose required during precrosslinking with varying concentrations of CM, GM and HM (% w/v). The solution was precrosslinked using blue light at an intensity of 114 mW/cm². The exact amount of blue light needed to precrosslink the biomaterials is shown inside each box.

FIG. 8 shows the precursor solution with or without APS/TEMED after blue light irradiation. The precursor solution was irradiated for 3 minutes with pulsed blue light (2.5 s on/2.5 s off) which had an energy flux of 114 mW/cm². The precursor solution without APS/TEMED remained a liquid after 3 minutes of blue light irradiation (left) whereas the precursor solution with APS/TEMED transformed into a hydrogel (right).

FIG. 9 shows hydrogel swelling with varying concentrations (w/v %) of peptide, chondroitin methacrylate (CM), and hyaluronic acid methacrylate (HM) on hydrogel swelling. Hydrogels were swollen for 24 h in saline solution with 5% wt/wt dextran. Hydrogels were synthesized without employing the APS/TEMED crosslinking system.

FIG. 10 shows cryo-scanning electron microscopy (Cryo-SEM) of G44 and G50 hydrogels. The pore size was determined using ImageJ software calculated from measuring +100 individual pores per sample from independent regions of the hydrogels.

FIG. 11 shows that peptide-based materials have suitable physical and biocompatible properties as intrastromal corneal bulking agents. (A) Top: Viscosity as a function of shear rate (s⁻¹) measured for the four different peptide-based formulations. Bottom: Compression moduli for fully crosslinked materials (n≥3). Data showed the plot are represented as box plots where the box encloses 50% of the data, upper and lower quartile, with the median value of the variable displayed as a line inside the box. The bars extending from the top and bottom of each box mark the minimum and maximum values within the data set that fall within an acceptable range. (B) Top: Schematic depiction for the main steps used in the ex vivo testing of the injectable materials (all samples were in situ crosslink 2.5 J/cm²). Bottom left: Average thickness of the injected formulations measured from OCT images at different time points post-injection (n=3). Bottom right: Average thickness of the injected G44 formulations using different volumes (10, 30, and 50 μL). Thickness was measured using OCT 48 h post injection (n≥3). (C) Left: Reduction in porcine cornea steepness after intrastromal injection of photoactivated material and blue light irradiation with keratoconus lenses to maintain desired shape. Right: Representative images of cornea pre-op and 24 h post-surgery. Dashed white line outlines anterior and posterior corneal curvature. Scale bars: 1 mm. (D) Number of live human-corneal epithelial cells per field of view (FOV) measured after 48 h seeding on pre-made peptide-based formulations hydrogels (n≥3).

FIG. 12 shows corneal stromal cell density of porcine corneas at different time points following intrastromal injections. Cell density was determined by counting the number of cells within sections of confocal microscopy images using FIJI. Unoperated corneas and corneas injected with Viscoat were tested as controls.

FIG. 13 shows differential scanning calorimetry (DSC) curves of cornea samples (controls and photocrosslinked) taken after 48 h at normal intraocular pressure within the perfusion chamber. An endothermic peak representing the denaturation temperature of the corneas was present around 69° C. for all groups.

FIG. 14 shows the change in porcine cornea transmittance after creation of the intrastromal pocket and material injection. The transmittance was measured over the entire visible spectrum.

FIG. 15 shows the transmittance of keratoconus lenses to UV (365 nm) and blue light (460 nm). The transmittance of the hard contact lenses to blue light was about 3 orders of magnitude higher than that of UV light.

FIG. 16 shows hematoxylin and eosin (H&E) staining of porcine corneas following intracorneal injections. An unoperated cornea (A) and cornea injected with Viscoat (B) were used as controls. Photoactivated materials G44 (C), G50 (D), G64 (E), and G65 (F) were injected into stromal pockets, respectively, and crosslinked using blue light. Approximately 50 μL of material was injected into each porcine cornea.

FIG. 17 shows the performance of peptide-based materials as corneal bulking agent in rats. (A) Schematic illustrating the protocol used to thicken rat corneas in vivo. (B) Peptide-based materials do not induce corneal opacity or promote visible vascularization. Cornea transparency was monitored after biomaterial intrastromal injection over 6-week period. Hydrogel-filled corneas exhibited similar transparency to pre-operated corneas and biomaterial injection sites resulted in minimal corneal scarring. (C) Peptide-based materials remained stable overtime after intracorneal injection in a rat model. OCT images of rat corneas (G44-A and B; G50-D and F) at different time points before and after surgery illustrate retention of the injected hydrogels within the corneal stroma 6 weeks post operation. In two corneas (G44-C and G50-E) the injected hydrogel was lost shortly after surgery. Higher resolution in vivo OCT images (FIG. S16) were obtained at the end of the study. They confirmed with greater detail the anatomy of the injected corneal tissue and implants. (D) Histology of intracorneal injection in a rat model. Hematoxylin and eosin staining shows the retention of the changed shape of the cornea in two rats after injection with the bulking agents (G44-A and G50-D). In the same two corneas, picrosirius red and alcian blue confirmed the presence of the glycosaminoglycan components of the hydrogels. The bright Picrosirius red fluorescence clearly shows the red of the rat corneal stromal layers around the bulking agent. The arrows in the G50-D treated corneas show incorporation of hydrogel into the spread corneal lamellae. The unoperated corneas had normal histology as shown by the hematoxylin and eosin, picrosirius red, and alcian blue. All images were processed in FIJI. Scale bars: 50 μm.

FIG. 18 shows representative images for LIVE/DEAD assay of corneal epithelial cells cultured onto treated well plate and fully formed hydrogels. Scale bars correspond to 100 μm in all cases.

FIG. 19 shows that peptide-based materials do not induce corneal opacity or promote visible vascularization. Cornea transparency was monitored after biomaterial intrastromal injection over 6-week period. Hydrogel-filled corneas exhibited similar transparency to preoperated corneas and biomaterial injection sites resulted in minimal corneal scarring.

FIG. 20 shows Thorlabs OCT imaging which confirmed that hydrogel formulations remained stable inside rat corneas. OCT images of rat corneas (G44-A and B; G50-D and F) at the end of the study showed that the injected hydrogels were preserved within corneal stroma. In two corneas (G44-C and G50-E), the injected hydrogel was lost soon after intracorneal surgery.

FIG. 21 shows that peptide-based materials remained stable overtime after intracorneal injection in a rat model. OCT images of rat corneas (G44-A and B; G50-D and F) at different time points before and after surgery illustrate retention of the injected hydrogels within the corneal stroma 6 weeks post operation. In two corneas (G44-C and G50-E) the injected hydrogel was lost shortly after surgery.

FIG. 22 shows Control rat corneas after intraocular surgery. Scarring was visible in each of the control rat corneas after the operation (A). Viscoat remained inside the cornea pocket immediately after intrastromal injection into the cornea but fully disappeared after three days (B).

FIG. 23 shows histology of intracorneal injection in a rat model for G64 and G65. The unoperated cornea had normal histology in the hematoxylin and eosin (h) and picrosirius red and alcian blue (o,v). All images were processed in FIJI. Scale bar: 50 μm.

FIG. 24 shows NMR spectra for the three acrylated natural polymers used in biomaterial preparation. The peaks pertaining to the acrylate groups on the polymers are identified by the black arrows. The presence of acrylate groups was verified for hyaluronic acid glycidyl methacrylate (HM), chondroitin glycidyl methacrylate (CM) and gelatin methacrylate (GM).

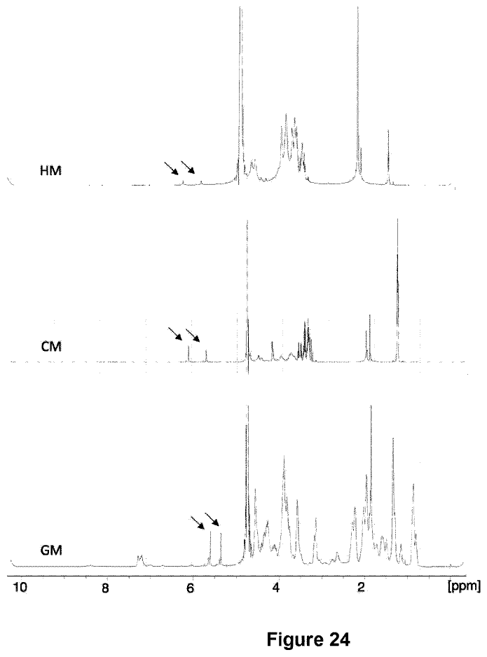

FIG. 25 shows blue light irradiation of riboflavin-containing formulations under different environmental conditions. Riboflavin sample 587 irradiated under 1% oxygen formed a hydrogel after 20 minutes (left) whereas riboflavin sample 588 irradiated under nitrogen did not completely crosslink and remained a semi-liquid (right).

FIG. 26 shows pachymetry maps pre- and post-surgery. Maps 1 and 2 demonstrate that porcine corneas are thicker than human corneas (pink color above the standard threshold for human corneas). Map 3 shows difference map showing the increase in pachymetry generated by the photocrosslinked implant. The blue marks show that the central pachymetry increased by 190 μm (from 1013 to 1203 μm). Distribution of the thickening however is not uniform. Map 4 shows Axial/Sagittal curvature difference map that displays an asymmetry of the front corneal surface curvature, which is flatter (−16.3 Diopters) over the inferior thicker zone of the implant and steeper (+22.5 Diopters) on the opposite side of the cornea.

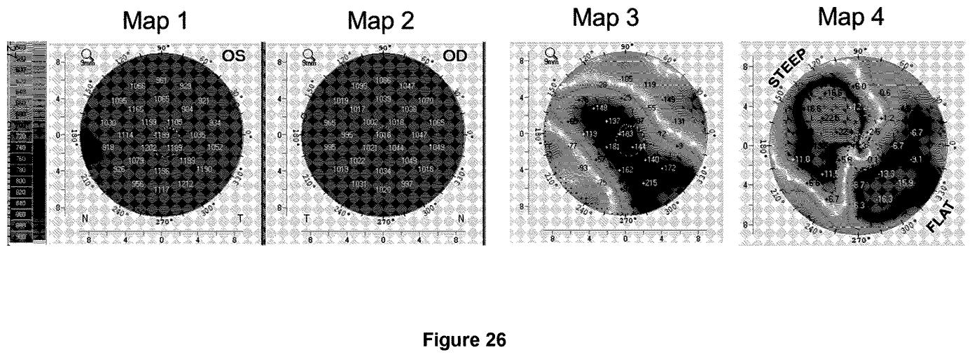

### DETAILED DESCRIPTION

Novel peptides, scaffolds, hydrogels, and methods of treating corneal diseases using the novel peptides, scaffolds and hydrogels will be described hereinafter. The use of novel peptides, scaffolds, and hydrogels for treating corneal diseases will also be described hereinafter. Although the invention is described in terms of specific illustrative embodiments, it is to be understood that the embodiments described herein are by way of example only and that the scope of the invention is not intended to be limited thereby.

The terminology used herein is in accordance with definitions set out below.

By “about”, it is meant that the value can vary within a certain range depending on the margin of error of the method or device used to evaluate or measure. A margin of error of 10% is generally accepted.

The description which follows, and the embodiments described therein are provided by way of illustration of an example of particular embodiments of principles and aspects of the present invention. These examples are provided for the purposes of explanation and not of limitation, of those principles of the invention. In the description that follows, like parts and/or steps are marked throughout the specification and the drawing with the same respective reference numerals.

To safely and non-invasively thicken a cornea and stabilize the collagenous matrix, the inventors tested the hypothesis photocuring using low energy pulsed light irradiation will mitigate cytotoxic effects of continuous irradiation and can therefore be used to safely photo-crosslink photosensitive and water-retaining ECM-mimetic materials designed for injection into thinning corneas. Also, in engineering our material, the inventors have ex vivo evaluated the suitability of our technology to thicken pig-corneas while using rigid contact lenses, which are prescribed in early stages of keratoconus. Further, as keratoconus patients' corneas vary in their thickness (22), the materials developed herein were ex vivo tested in pig corneas by using different injection volumes to produce implants of varied thickness.

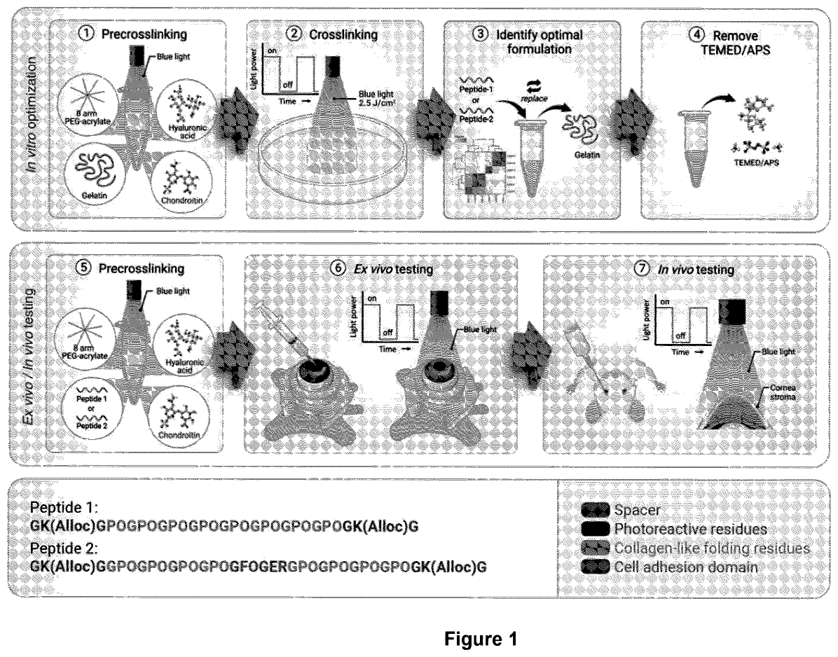

The research strategy followed is summarized in FIG. 1. At the outset, GelMA was used to build a library of biomaterial formulations with differing mechanical properties. Soft biomaterials were created that can intercalate into and thicken a slightly thinned stroma (>450 μm) to robust hydrogels that retain their shape and significantly increase the bulk of a very thin stroma (<450 μm). Then, for actual in vivo crosslinking in animal models, the GelMA was substituted with one of two different collagen-like peptides (CLP) with and without the GFOGER (SEQ ID NO: 1) cell adhesion motif (FIG. 1B). The candidate materials were assessed in vitro for toxicity and ex vivo within excised pig corneas to determine their ability to retain their shape over 24 h under intraocular pressure. The most promising candidates were then injected into rat corneas using a minimally invasive procedure, and the animals were monitored for up to 6 weeks.

### Photosensitive Bulking Agent (PBA), Scaffolds, Precursor Hydrogels, Hydrogels, Compositions Thereof Uses Thereof and Methods Thereof

Described herein are photosensitive bulking agents, scaffolds, precursor hydrogels, hydrogels, compositions thereof as well as uses and methods for the production thereof. Methods of treating corneal diseases are also described herein. It will be appreciated that embodiments and examples are provided for illustrative purposes intended for those skilled in the art, and are not meant to be limiting in any way.

In an embodiment, there is provided herein a photosensitive bulking agent comprising a photoreactive collagen-like peptide (PCLP) and polyethylene glycol (PEG). Although collagen has been widely used as biomedical materials for tissue support and regeneration due to excellent gel-forming abilities, its thermal instability and potential contamination with pathogenic substances have lead to the use of synthetic model collagens, also known as collagen-like peptides (CLPs) or collagen-mimetic peptides (CMPs), in these types of applications. Collagen-like peptides (CLPs), also known as collagen-mimetic peptides (CMPs), include short synthetic peptides that mimic the triple helical conformation of native collagens. PEG is also known as polyethylene oxide (PEO) or polyoxyethylene (POE), depending on its molecular weight. PEG is widely used in medicine as an excipient in many pharmaceutical products as it is considered biologically inert and safe by the FDA. The PEG of the present invention may be a 2-Arms-PEG acrylate, 4-Arms-PEG acrylate, or an 8-Arms-PEG acrylate, preferably an 8-Arms-PEG acrylate.

As will be understood, the scaffold biomaterial may, in certain embodiments, comprise riboflavin, also known as vitamin B2. Riboflavin acts as a photosensitizer such that when exposed to light, riboflavin can generate reactive oxygen species that induce the formation of covalent bonds both between collagen molecules and between collagen molecules and proteoglycans and may protect the endothelium that is UV sensitive.

In an embodiment, the PCLP comprises at least one photoreactive residue. Examples of photoreactive amino acid residues include tryptophan, tyrosine, phenylalanine, cysteine, lysine, photoreactive diazirine analogs of leucine and methionine, and para-benzoylphenylalanine.

In an embodiment, each of the at least one photoreactive residue may be flanked by two aliphatic amino acid residues selected from the group consisting of glycine residues, alanine residues, valine residues and a combination thereof. In certain embodiments, the two aliphatic amino acid residues may be glycine residues.

In an embodiment, the PCLP comprises a plurality of collagen-like folding motifs. Collagen has an unusual amino acid composition and sequence with glycine found at almost every third residue and proline making up about 17% of the protein. Moreover, collagen contains two uncommon derivative amino acids not directly inserted during translation, hydroxyproline derived from proline and hydroxylysine derived from lysine, both requiring vitamin C as a cofactor. The most common motifs in the amino acid sequence of collagen are glycine-proline-X and glycine-X-hydroxyproline, where X is any amino acid other than glycine, proline or hydroxyproline. Examples of collagen-like folding motifs include GPO, POG, RGD, GROGER (SEQ ID NO:2), GLOGEN (SEQ ID NO:3), and GMOGER (SEQ ID NO:4).

In certain embodiments, the PCLP comprises at least one cell adhesion motif flanked by the plurality of collagen-like folding motifs. Examples of cell adhesion motifs include RGD(S), PHSRN, and REDV derived from fibronectin; YIGSR and IKVAV derived from laminin; and DGEA, GFOGER/GFPGER derived from collagen.

In an embodiment, the at least one cell adhesion motif may comprise the amino acid residues GFOGER (SEQ ID NO:1).

In an embodiment, the PBA is sensitive to light, preferably light having low energy in order to minimize toxicity. Typically, the human eye can detect wavelengths from 380 to 700 nanometers. Although small does of ultraviolet light may be used, photons within the visible spectrum are preferred, as these have lower energy and are thus less toxic. In certain embodiments, the visible light has a wavelength of about 400 nm to about 490 nm and may be blue light. In certain embodiments, the blue light has a wavelength of about 460 nm.

In an embodiment, the PCLP comprises the amino acid sequence defined by (X₀)ₘX₁(X₆)ₙ(X₂)ₒ(X₇)ₚX₅(X₈)_{q}. Each of X₀, X₆, X₇, and X₈ may independently represent a glycine, an alanine, or a valine, wherein 1≤m≤6, 1≤n≤6, 1≤q≤6 and 0≤p≤6. X₁ and X₅ may be photoreactive residues which may be the same or different. X₂ may represent collagen-like folding motifs, wherein 3≤o≤20.

In an embodiment, each of X₁ and X₅ represent a lysine, cysteine, tryptophan, tyrosine, or histidine protected by a N-allyloxycarbonyl (Alloc) group, a 9-fluorenylmethoxycarbonyl (Fmoc) group or a tert-butyloxycarbonyl (Boc) group. Other examples of protecting groups may be used while still remaining within the scope of the invention. In certain embodiments, each of X₁ and X₅ represent a lysine protected by a N-allyloxycarbonyl (Alloc) group.

In an embodiment, X₂ is defined by amino acid sequence GPO, POG, RGD, GROGER (SEQ ID NO:2), GLOGEN (SEQ ID NO:3), or GMOGER (SEQ ID NO:4). In certain embodiments, X₂ is defined by amino acid sequence POG. o=4. In certain embodiments, each of X₀, X₆, X₇, and X₈ represent a glycine. each of m, n, and q=1 and p=0.

In an embodiment, the PCLP comprises the amino acid sequence defined by GK(Alloc)GPOGPOGPOGPOGPOGPOGPOGPOGK(Alloc)G (SEQ ID NO:5), wherein amino acid residue K(Alloc) represents a lysine protected by a N-Allyloxycarbonyl (Alloc) group.

In an embodiment, the photoreactive collagen-like peptide (PCLP) comprises the amino acid sequence defined by (X₀)ₘX₁(X₆)ₙ(X₂)ₒX₃(X₄)ₚ(X₇)_{q}X₅(X₈)ᵣ. Each of X₀, X₆, X₇, and X₈ may independently represent a glycine, an alanine, or a valine, wherein 1≤m≤6, 1≤n≤6, 1≤q≤6, and 1≤r≤6. X₁ and X₅ may be photoreactive residues which are the same or different. X₂ and X₄ may be collagen-like folding motifs, wherein X₂ and X₄ are the same or different and 2≤o≤10 and 2≤p≤10. X₃ may represent at least one cell adhesion motif Each of X₁ and X₅ may represent a lysine, cysteine, tryptophan, tyrosine, or histidine protected by a N-allyloxycarbonyl (Alloc) group, a 9-fluorenylmethoxycarbonyl (Fmoc) group or a tert-butyloxycarbonyl (Boc) group. Each of X₁ and X₅ may represent a lysine protected by a N-allyloxycarbonyl (Alloc) group. Each of X₂ and X₄ may be defined by amino acid sequence GPO, POG, RGD, GROGER (SEQ ID NO:2), GLOGEN (SEQ ID NO:3), or GMOGER (SEQ ID NO:4). In certain embodiments, each of X₂ and X₄ is defined by amino acid sequence GPO. In certain embodiments, each of o and p=4. Each of X₀, X₆, X₇, and X₈ may represent a glycine. In certain embodiments, each of m, n, q, and r=1.

In an embodiment, the PCLP comprises the amino acid sequence defined by GK(Alloc)GGPOGPOGPOGPOGFOGERGPOGPOGPOGPOGK(Alloc)G (SEQ ID NO:6), wherein amino acid residue K(Alloc) represents a lysine protected by a N-allyloxycarbonyl (Alloc) group.

In an embodiment, the PBA may be a photosensitive corneal stromal bulking agent.

In an embodiment, there is provided herein a composition comprising the PBA as described herein and a physiologically acceptable carrier.

In an embodiment, there is provided herein a scaffold comprising the PBA as described herein. The scaffold may further comprise hyaluronic acid methacrylate and/or chondroitin methacrylate. Naturally occurring glycosaminoglycans (GAGs), such as chondroitin sulfate (CS) and hyaluronic acid (HA), are very attractive materials for designing biomimetic hydrogels as CS is a sulfated linear polysaccharide composed of glucuronic acid and N-acetylgalactosamine as its repeating disaccharide unit. HA is also a linear polysaccharide, whose disaccharide repeating unit is composed of glucuronic acid and N-acetylglucosamine. Both polymers are highly hydrophilic, negatively charged, and, therefore, characterized by water retention capacity and possessed of specific rheological, physiological, and biological properties. Hyaluronic acid-based hydrogels are widely used in tissue engineering, 3D bioprinting, and drug delivery applications. The methacrylate-functionalized hyaluronic acid is photo-crosslinkable and can be used to generate crosslinked hydrogels.

In an embodiment, there is provided herein a precursor hydrogel obtained by crosslinking a precursor solution comprising the PBA as described herein. The precursor hydrogel may further comprise hyaluronic acid methacrylate and/or chondroitin methacrylate. The precursor hydrogel may be obtained by crosslinking the precursor solution by irradiating the precursor solution with visible light in vitro. The visible light may have a wavelength of about 400 nm to about 490 nm. The visible light may be blue light having a wavelength of about 460 nm.

In an embodiment, the precursor hydrogel may have a viscosity within about 10% of the viscosity of Viscoat®. Viscoat® is a combination of sodium hyaluronate 3% and chondroitin sulfate 4%, has the typical properties of a dispersive ophthalmic viscosurgical devices (OVDs).

In an embodiment, the precursor hydrogel may be for use in preparing a hydrogel, in intrastromal injection, in treating a corneal disease, in treating a corneal disease characterized by a thinning cornea, in combination with a cornea implant for treating a corneal disease, or any combination thereof. Examples of diseases characterized by a thinning cornea include keratitis and corneal dystrophies such as keratoconus, Fuch's dystrophy, lattice dystrophy, and Map-dot-fingerprint dystrophy.

In an embodiment, there is provided herein a hydrogel comprising the precursor gel as described herein which may be obtained by crosslinking the precursor gel in vivo. Crosslinking the precursor gel in vivo may be performed by irradiating a subject injected with the precursor solution. The hydrogel may be obtained by irradiating the precursor gel with visible light having a wavelength of about 400 nm to about 490 nm. The visible light may be blue light having a wavelength of about 460 nm.

In an embodiment, the hydrogel comprises pores having a diameter of between about 10 μm to about 50 μm.

In an embodiment, the hydrogel has a compression modulus within about 10% of the compression modulus of a mammalian cornea.

In an embodiment, the hydrogel has a shear rate of between about 0 s⁻¹ to about 200 s⁻¹.

In an embodiment, the crosslinking allows conjugation of the photoreactive collagen-like peptide (PCLP) with the polyethylene glycol (PEG).

In an embodiment, there is provided herein a method of preparing a hydrogel in vivo. The method comprises providing a precursor solution comprising a photoreactive collagen-like peptide (PCLP), polyethylene glycol (PEG) and riboflavin. The method further entails precrosslinking the precursor solution by irradiating the precursor solution with visible light to obtain a precursor hydrogel; injecting the precursor gel at a desired location in a subject; and crosslinking the precursor gel by irradiating the desired location in the subject with visible light to obtain a hydrogel in the subject.

In an embodiment, the photoreactive collagen-like peptide (PCLP) comprises at least one photoreactive residue. Each of the at least one photoreactive residues may be flanked by two glycine residues.

In an embodiment, the photoreactive collagen-like peptide (PCLP) comprises a plurality of collagen-like folding residues.

In an embodiment, the photoreactive collagen-like peptide (PCLP) comprises at least one cell adhesion motif flanked by the plurality of collagen-like folding residues.

In an embodiment, the precursor solution comprises hyaluronic acid methacrylate, gelatin methacrylate, chondroitin methacrylate or any combination thereof.

In an embodiment, the visible light has a wavelength of about 400 nm to about 490 nm.

In an embodiment, the visible light is blue light. The blue light may have a wavelength of about 460 nm.

In an embodiment, the precrosslinking and/or crosslinking are performed under low oxygen concentration. The low oxygen concentration may be about 1% v/v.

In an embodiment, precrosslinking the precursor solution may be performed at 114 mW/cm².

In an embodiment, the precrosslinked precursor solution is centrifuged to remove bubbles therein.

In an embodiment, irradiating the precursor solution and/or irradiating the desired location in the subject is performed by pulsed irradiation.

In an embodiment, the pulsed irradiation comprises irradiating for about 0.5 s to about 5 seconds at intervals of about 0.5 s to about 5 seconds.

In an embodiment, a total light dosage irradiated at the desired location in the subject is about 2.5 J/cm².

In an embodiment, the PCLP comprises at least one cell adhesion motif.

In an embodiment, the hydrogel has a shear rate of between about 0 s⁻¹ to about 200 s⁻¹.

In an embodiment, the photoreactive collagen-like peptide (PCLP) comprises the amino acid sequence defined by (X₀)ₘX₁(X₆)ₙ(X₂)ₒX₃(X₄)ₚ(X₇)_{q}X₅(X₈)ᵣ. Each of X₀, X₆, X₇, and X₈ may independently represent a glycine, an alanine, or a valine, wherein 1≤m≤6, 1≤n≤6, 1≤q≤6, and 1≤r≤6. X₁ and X₅ may be photoreactive residues which are the same or different. X₂ and X₄ may be collagen-like folding motifs, wherein X₂ and X₄ are the same or different and 2≤o≤10 and 2≤p≤10. X₃ may represent at least one cell adhesion motif.

In an embodiment, each of X₁ and X₅ represent a lysine, cysteine, tryptophan, tyrosine, or histidine protected by a N-allyloxycarbonyl (Alloc) group, a 9-fluorenylmethoxycarbonyl (Fmoc) group or a tert-butyloxycarbonyl (Boc) group. In certain embodiments, each of X₁ and X₅ represent a lysine protected by a N-allyloxycarbonyl (Alloc) group.

In an embodiment, each of X₂ and X₄ is defined by amino acid sequence GPO, POG, RGD, GROGER (SEQ ID NO:2), GLOGEN (SEQ ID NO:3), or GMOGER (SEQ ID NO:4). In certain embodiments, each of X₂ and X₄ is defined by amino acid sequence GPO. In certain embodiments, each of o and p=4. In certain embodiments, each of X₀, X₆, X₇, and X₈ represent a glycine. In certain embodiments, each of m, n, q, and r=1.

In an embodiment, the PCLP comprises the amino acid sequence defined by GK(Alloc)GGPOGPOGPOGPOGFOGERGPOGPOGPOGPOGK(Alloc)G (SEQ ID NO:6), wherein amino acid residue K(Alloc) represents a lysine protected by a N-allyloxycarbonyl (Alloc) group.

In an embodiment, the hydrogel has a viscosity within about 10% of the viscosity of Viscoat®.

In an embodiment, the hydrogel has compression modulus within about 10% of the compression modulus of a mammalian cornea.

In an embodiment, crosslinking the precursor gel is performed by irradiating the desired location in the subject with visible light for a period of between about 2 minutes to about 15 minutes.

In an embodiment, there is provided herein a cornea implant comprising the precursor hydrogel as described herein or the hydrogel as described herein.

In an embodiment, there is provided herein a method of treating a condition of the eye characterized by a corneal defect. The method may comprise making a limbal incision in an area affected by the corneal defect in a subject; administering the precursor hydrogel over the corneal defect; and irradiating the precursor hydrogel with visible light.

In an embodiment, there is provided herein a peptide comprising the amino acid sequence GK(Alloc)GPOGPOGPOGPOGPOGPOGPOGPOGK(Alloc)G (SEQ ID NO:5), wherein amino acid residue K(Alloc) represents a lysine protected by a N-allyloxycarbonyl (Alloc) group.

In an embodiment, there is provided herein a peptide comprising the amino acid sequence GK(Alloc)GGPOGPOGPOGPOGFOGERGPOGPOGPOGPOGK(Alloc)G (SEQ ID NO:6), wherein amino acid residue K(Alloc) represents a lysine protected by a N-allyloxycarbonyl (Alloc) group.

In an embodiment, there is provided herein a composition comprising the peptide as described herein and an 8-Arms-PEG acrylate. In certain embodiments, the composition may not be crosslinked. In certain embodiments, the composition may be partially crosslinked. In certain embodiments, the composition may be fully crosslinked.

In an embodiment, there is provided herein a use of the composition as described herein for treating, preventing, or augmenting corneal thinning or any other corneal defects in a subject in need thereof.

### Examples

### Materials and Methods

The following material and methods were performed in the examples described below.

### Materials

Hyaluronic acid sodium salt from Streptococcus equi, chondroitin sulfate A sodium salt from bovine trachea, gelatin from porcine skin type A (bloom 175), polyethylene glycol diacrylate (MW 250 g/mol and MW 700 g/mol), 8-arm polyethylene glycol acrylate (MW 20,000 g/mol), and glycidyl methacrylate, riboflavin, APS and TEMED were purchased from Sigma-Aldrich. HEPES was purchased from Fisher-Scientific. Dextran sulfate sodium salt was purchased from USB corporation.

### Synthesis of Hyaluronic Acid Glycidyl Methacrylate

Methacrylated hyaluronic acid was prepared similarly to the previously developed protocol (56). Briefly, 500 mg of hyaluronic acid was dissolved in 100 mL 1× phosphate buffer saline and 33.5 mL of dimethylformamide. Then, 6.58 mL of glycidyl methacrylate and 4.61 mL of TEMED were mixed into the solution dropwise and the methacrylation reaction was carried out for 10 days at 25° C. The reaction solution was precipitated twice in 2 L of acetone, vacuum filtered, dialyzed against milliQ water for 3 days, and lyophilized for 3 days, before being stored at −20° C. Successful acrylation of hyaluronic acid was verified by proton nuclear magnetic resonance (NMR) spectroscopy in D₂O (FIG. S20).

### Synthesis of Chondroitin Glycidyl Methacrylate

Chondroitin sulfate was methacrylated with glycidyl methacrylate based on the published protocol (57). Chondroitin sulfate was dissolved in a 50:50 mixture of acetone and ddH₂O forming a 1 w/v % solution of chondroitin sulfate. Once the chondroitin sulfate was fully dissolved in acetone/ddH₂O, a 20-fold molar excess of triethylamine (TEA) per chondroitin sulfate was added to the solution. After adding TEA, a 20-fold molar excess of glycidyl methacrylate per chondroitin sulfate was added dropwise to the mixture using a syringe pump set at 10 mL/min. The solution was stirred at room temperature for one day to allow the methacrylation reaction to progress. The resulting products were dialyzed in 50:50 acetone:water for 24 h (14 kDa MWCO membrane) and then in milliQ water for 2 days. The methacrylated chondroitin sulfate was lyophilized for 4 days and stored at −20° C. prior to use. Methacrylation of chondroitin sulfate was verified by NMR in D₂O to determine the degree of conjugation of the glycidyl methacrylate groups to chondroitin sulfate (FIG. S20).

### Synthesis of Gelatin Methacrylate

Gelatin methacrylate was prepared based on the pre-established protocol (58). Type A gelatin (175 bloom) from porcine skin was dissolved in 0.25 M of carbonate-bicarbonate buffer (final pH 9.0) to prepare a 10 w/v % gelatin solution. Next, methacrylic anhydride (94%) was added to the gelatin solution (0.2 mL MAA/g gelatin) while stirring with a magnetic stir bar at 500 rpm. After 3 hours, the pH of the solution was adjusted to 7.4 to stop the reaction. The gelatin methacrylate was filtered and dialyzed using a 14 kDa molecular weight cut off cellulose membrane for 3 days. The purified product was dried in a lyophilizer and then stored at −20° C. until use. Methacrylation of gelatin was verified by proton NMR (FIG. S20).

### Peptide Synthesis

Peptides 1 and 2 were synthesized using the Liberty Blue (CEM) automated microwave peptide synthesizer. Fmoc protected amino acids were purchased from CEM. To glycine preloaded Wang resin, Fmoc deprotection was carried out with 20% piperidine at 90° C. for 60 s. Customized DIC/Oxyma coupling cycles with increased temperature, reaction time, reagent equivalents, and addition of chaotropic agent were performed at 0.1 mmol scale. Peptides were removed from the resin and deprotected with 92.5/2.5/2.5/2.5% v/v TFA/TIS/EDT/H2O at 37° C. for one hour and then precipitated with −20° C. diethyl ether in a Falcon tube. After chilling in a −20° C. freezer for 30 minutes or overnight, peptides were centrifuged at 5000 rpm for 4 min. The ether supernatant was discarded, and the crude precipitate was dried under vacuum. Reversed-phase high performance liquid chromatography (RP-HPLC) was then used to purify each peptide with a Luna 5 m C18 100 Å 250×21.2 mm column and two solutions of 94.9% water 5% acetonitrile (MeCN) 0.1% TFA (A) and 99.01% MeCN 0.09% TFA (B) to generate a gradient. Peptides were obtained at >95% purity. Mass spectrometry confirmed the presence of peptide 1 (899 M+3H+) and peptide 2 (1157 M+3H+). Selected HPLC fractions were lyophilized to leave a fluffy, white solid consisting of pure peptide.

### Hydrogel Synthesis

The hydrogel precursor solution was prepared with 9% w/v 8-arm PEG acrylate, 3% w/v PEG diacrylate (Mn 700 g/mol), 0.6% w/v PEG diacrylate (Mn 250 g/mol) 10 mM of HEPES pH 7.2 and 0.14 mM riboflavin. In the case of APS/TEMED hydrogels, 1 mM of TEMED and APS were added instead of HEPES buffer. Depending on the test, hyaluronic acid methacrylate, gelatin methacrylate, and chondroitin methacrylate may also have been added to the precursor solution. The precursor solution was mixed at max speed for 10 minutes until all components were equally dispersed in the solution. In the case of APS/TEMED hydrogels, APS was added dropwise to the precursor solution to achieve a final concentration of 1 mM and then the solution was mixed for an additional 5 minutes. After mixing, the precursor solution was crosslinked under 1% oxygen using light until the stir bar was only able to vibrate slowly in solution (gelation point). The intensity of the blue light for precrosslinking was 114 mW/cm². Next, the precrosslink solution was taken up using a syringe and centrifuged at 5000 rpm for 10 min to remove bubbles. The biomaterial was then transferred into a 96-well plate for crosslinking in vitro, injected into pig corneas for ex vivo crosslinking or injected into rat corneas for in vivo crosslinking. The biomaterial was crosslinked using blue light at 8.5 mW/cm² for 10 min (2.5 J/cm² total energy dose). The calculation of the total energy dose can be found in the supplementary information. Crosslinking in vitro was done inside a sealed, humid container with 1% O₂ to simulate the cornea's natural environment.

### Swelling

After crosslinking, the finished hydrogel was blotted gently with a Kimwipe™, weighed, and then placed in 1×PBS or saline solution (5% dextran) at 37° C. for 24 h. The hydrogel was removed from PBS/saline solution after 24 h, and then weighed. The hydrogel was placed back in PBS/saline solution and allowed to swell for an additional 7 days. The swelling was calculated by taking the difference between the mass of the swollen hydrogel and the mass of the hydrogel before swelling, and then dividing the difference by the mass before swelling.

### Equilibrium Water Content

After swelling in saline solution (5 wt % dextran), hydrogels were weighed and then dried at room temperature for 8 h. Then, the dry mass of each hydrogel was recorded, and the equilibrium water content was calculated by taking the difference between the swollen and dry hydrogel masses and dividing by the swollen mass.

### Transparency

APS/TEMED hydrogels were allowed to swell for 7 days and then were placed individually into a 96-well plate. The 96-well plate containing the hydrogels was inserted into a multimode, microplate reader to measure the optical density of each hydrogel. The microplate reader measured the optical density of the hydrogel at 37° C. for the entire visible spectrum. The temperature was set to 37° C. since this is roughly the temperature of the human cornea. These transmittance measurements were slightly affected by variable hydrogel surface roughness causing reflection at the hydrogel-air interface. To limit the impact of diffuse reflection on light transmittance measurements, subsequent APS/TEMED-free hydrogels were immersed in 100 μL of saline/dextran solution before evaluating hydrogel transparency.

### Refractive Index

After allowing the newly made hydrogels to swell for 24 h in PBS solution, the hydrogels were removed from the PBS solution and placed, one at a time, in a refractometer. The refractometer measured the refractive index of each hydrogel at 37° C. [Anton-Paar/Abbemat 300].

### Viscosity

After sol formation, the viscosity of each biomaterial formulation was measured using a Brookfield rheometer with an RCT-25-1 spindle. The micrometer ring of the rheometer was first set to zero and then the measuring head was lowered to the zero position. The spindle was tightened and then the micrometer was set to the measuring point (M) allowing for 50 μm of space between the spindle and the bottom plate. The biomaterial (100 μL) was loaded onto the center of the plate and the spindle was lowered to the M position compressing the sample. Excess sample was removed and then the test was executed. The viscosity was measured between 0-200 s 1 shear rate at 37° C. using Rheo3000 v2 software.

### Differential Scanning Calorimeter

Hydrogels were analyzed using a differential scanning calorimeter (TA Instruments/Q200). Each hydrogel was blotted gently with a Kimwipe™, placed in an aluminum hermetic pan and sealed with an aluminum hermetic lid to prevent evaporation of water. To measure the water distribution of each hydrogel, the temperature ranged from −50° C. to 210° C. with a cooling/heating rate of 10° C./min. The mass of each of these samples was between 0.7-6.0 mg. The proportion of water in each hydrogel was determined using DSC results as described in the supplementary information. To determine the glass transition temperature of the top hydrogel candidates, hydrogels were heated from 10° C. to 90° C. at a rate of 10° C./min.

### Collagenase Degradation Assay

Collagenase from Clostridium histolyticum (Sigma-Aldrich) at 5 U/mL in 0.1 M Tris-HCl buffer pH 7.4 containing 5.0 mM CaCl₂ was used to evaluate the stability of the hydrogels as previously described. In summary, samples were blotted gently to remove surface water and weighed at different time points: 0, 15, and 24 h. Degradation rate was calculated using the slope of the plot in mass loss vs. time in h.

### Compression Strength

The compression strengths of the hydrogels and pig corneas were measured using an Instron 3342 with a 10 N load cell. The diameter of each sample was measured before executing each test. Swollen hydrogels and pig corneas (2-6 mm diameter) were placed one at a time on the center of the Intron's metallic platen and then the compression head was lowered slowly until it contacted the sample. Before starting each program, the load and distance were zeroed. The crosshead speed was set to 1 mm/min. The data was recorded using Series IX software. After testing, the actual thickness of each sample was calculated by taking the difference between the height at which the Instron first detected a significant resistance force and the height at which the sample was fully compressed. Then, the compression modulus was taken as the slope of the stress-strain plot between 0-10% deformation.

### Cryo-Scanning Electron Microscopy (Cryo-SEM)

Hydrogel samples were initially swollen in saline solution with 5% wt/wt dextran. Then, the microstructure of the hydrogels was evaluated using a TESCAN scanning electron microscope, Model Vega II XMU, at low-vacuum conditions. SEM images of hydrogels were captured at different magnifications. Setting conditions included an acceleration voltage of 20 kV, Cryo-Stage temperature around −50° C. and chamber vacuum around 35 Pa.

### Cell Preparation and Cell Seeding on Hydrogel Surface

Human corneal epithelial cells were cultured at 37° C. and 5% CO2 in Keratinocyte-SFM supplemented with human recombinant epidermal growth factor (rEGF), bovine pituitary extract (BPE), and 1% streptomycin/penicillin. Cells were grown in tissue culture treated flasks until approximately 70% confluence was reached. Then, hydrogels were prepared as previously described and washed in 1×PBS three times over a 24 h period. Hydrogels were then immersed in keratinocyte-SFM media for 1 h to acclimatize gels to the environment in a 96-well plate. After, the media was removed and 150 μL of corneal epithelial cell solution (6.2×10⁴ cells/mL) was added to the top of each hydrogel sample and, as a control, to the bottom of empty well plates. The 96-well plate was stored at 37° C. for 48 h before evaluating the cell viability.

### Cell Viability

The viability of human corneal epithelial cells was determined using a calcein acetoxymethyl (calcein AM) and ethidium homodimer-1 LIVE/DEAD assay from Invitrogen based on the instructions provided by the manufacturer. First, the staining solution was prepared by diluting the calcein AM (0.5 μL/mL) and ethidium homodimer-1 (2 μL/mL). Cell medium was removed from hydrogel-containing wells and replaced with 100 μL of staining solution. Cell-seeded hydrogels were then incubated for 15 min at 37° C. in the dark. Live (green stain) and dead (red stain) cells were imaged using an inverted fluorescent microscope from ZEISS (Axio Observer Z1) and cell viability was quantified by dividing the number of live cells by total number of cells, using ImageJ software.

### Ex Vivo Intrastromal Corneal Injections

Pig eyes were obtained from a certified slaughterhouse. Before cornea dissection, eyes were disinfected in Iodine-PVP (10%) for 2 minutes. Next, each eyeball was transferred to sterile 0.1% sodium thiosulphate solution for 1 minute and then immersed in sterile saline solution for 2 minutes. Using a scalpel blade, a small (5-8 mm) incision was made at the equatorial leaving approximately 8 mm of sclera around the cornea. Then, this incision was carefully extended by 360° around the entire eyeball while avoiding perforation of the underlying choroid layer. Once the cut was made across, the ciliary body-choroid was pulled downwards using forceps to avoid touching the corneo-scleral disk. The remaining posterior part of the eye was discarded, including lens, retina, and vitreous humour. The corneo-scleral disk was mounted on a perfusion chamber developed in house and sealed by compressing the scleral rim in-between the bottom and top parts of the perfusion chamber. The chamber was then filled with DMEM media with 5% dextran using two syringes attached to two irrigation ports that were then clamped to maintain an intracameral pressure within physiological range. A 6-mm biopsy punch was used to gently mark the central corneal surface. A 2 mm incision was then made at the edge of this mark to initiate the dissection of an intrastromal tunnel, which was carefully enlarged using a crescent knife to create an intrastromal pocket under the marked corneal surface. For intrastromal injections, each cornea was slightly deflated and 10 μL, 30 μL, or 50 μL of biomaterial was injected into the stromal pockets. Afterwards, the injected material was crosslinked using pulsed blue light with an intensity of 8.5 mW/cm² for 10 min. During crosslinking, the corneal surface was irrigated dropwise every 5 s with saline solution to prevent drying. Optical coherence tomography (OCT; Stratus, Carl Zeiss Meditec, Dublin, CA) and in vivo confocal microscopy (Confoscan; Nidek Technologies, Albignasego, Padova, Italy) imaging of the corneas were performed at four different time points during the procedure, namely after biomaterial injection, after crosslinking, 24 h post injection and 48 h post injection. When the corneas were not being evaluated by OCT nor Confocal microscopy, 3 mL of DMEM media with 5% dextran was added to completely cover the anterior corneal surface and the cornea chambers were stored in an incubator at 35° C.

### Ex Vivo Corneal Reshaping

Corneas were mounted on ex vivo perfusion chambers and biomaterials were injected as described previously. Keratoconic lenses (RGP and Centracone) were applied on top of corneas and sealed with a thin transparent film. Corneas were crosslinked using pulsed blue light with an intensity of 8.5 mW/cm² for 10 min. After crosslinking, lenses were removed and corneas were stored in DMEM media with dextran at 35° C. Optical coherence tomography (OCT; Stratus, Carl Zeiss Meditec, Dublin, CA) imaging of the corneas were performed at five different time points during the procedure, namely before pocket formation, after pocket formation, after biomaterial injection, after crosslinking, and 24 h post injection. Anterior corneal curvature was measured from OCT images by curve fitting over the central 3 mm diameter area in FIJI. To ensure cornea curvature measurements were not affected by corneal pressure fluctuations, the corneal pressure was measured and standardized for each test right before OCT imaging using a pressure sensor.

### Tissue Preparation, Histopathology, and Imaging of Pig Corneas

Following the dissection of the corneas, each cornea went through a sucrose gradient (5% to 20% sucrose), were incubated for 60 minutes in optimal cutting temperature medium (Tissue Tek O.C.T. compound, Sakura Finetek, USA), placed in isopentane, and frozen by liquid nitrogen. Each sample was sectioned at 16 or 18 m and placed on positively charged glass slides. The sections were stained with Hematoxylin and Eosin as well as with Picrosirius Red and Alcian Blue for histopathology examinations and then imaged using an Upright fully motorized widefield Zeiss imaging system at 10× magnification.

### Blue Light Toxicity in Feline Cornea

In this preliminary experimental study, we exposed one eye of two felines (n=2) to blue light (8.5 mW/cm2 for 10 min, 2.5 J/cm2 total energy dose). Pictures of both eyes were taken daily for a follow-up period of 8 days post-exposure to blue light, using several optical devices (slit lamp, Visante™ OCT, Thorlabs-OCT, Pentacam, specular microscope, Confoscan). The animals were sedated to ensure stillness and the human-designed devices exploited in this study were validated for usage in the context of our research. Moisturization drops were used to regularly hydrate the eyes throughout the procedure.

### In Vivo Intrastromal Corneal Injections

All in vivo experiments were conducted in accordance with the Canadian Council on Animal Care (CCAC) and the research protocol was approved by the Maisonneuve-Rosemont Hospital research center committee for animal protection. Nine Long Evans male rats (Charles River, Saint-Constant, QC, Canada) weighting 250 to 300 g were used for this study (6 eyes of 6 rats for testing the G44 and G50 formulations and 4 eyes of 3 rats for the controls). All surgeries were performed under general anesthesia by the same surgeon (AA). Animals were premedicated with a subcutaneous injection of buprenorphine (0.05 mg/kg; Vetergesic, Ceva, ON, Canada) and Lactated Ringer's solution (3 mL; Baxter Corporation, Mississauga, ON, Canada). Anesthesia was induced by inhalation of isoflurane 4% (Forane, Baxter) and maintained with isoflurane 1.5-2%. A thermal blanket was used to maintain the animal's temperature at 37° C. One eye per animal was randomly assigned to surgery and the contralateral eye was closed with ophthalmic gel (Ophtal Gel, Stom Pharm Inc, Montreal, QC, Canada). After disinfection with 0.5% iodine solution (Dovidine, Laboratoires Atlas, Montreal, QC, Canada), a sterile plastic film (Tegaderm fil, 3M HealthCare, St. Paul, MN, USA) was applied to the periocular region to stabilize the lids, recline lashes, and expose the eye. A 1-mm peripheral partial thickness corneal incision was made with a 30° micro knife (Beaver-Visitec, Waltham, MA), followed by an intrastromal tunnel (Dual Bevel 1.0 mm Angled Sideport Knife; Alcon, Fort Worth, TX), and a fine spatula was used to gently create an intrastromal pocket covering of the corneal area. The biomaterial was slowly injected using a 27G canula (Alcon), followed by OCT (Visante 1000; Carl Zeiss Meditec, Dublin, CA) imaging. The material was then crosslinked using pulsed blue light (cycle time 2.5 seconds on/2.5 seconds off), 8.5 mW/cm², for 10 min under dropwise saline irrigation. Postoperative medication consisted of a second subcutaneous dose of buprenorphine (8 to 12 hours post-surgery) and ophthalmic drops of prednisolone acetate (Teva prednisolone 1%, Teva Canada, Toronto, ON, Canada) and moxifloxacin 0.5% (Moxifloxacin, Sandoz, Boucherville, QC, Canada) twice a day until the end of the study. The animals were housed in enriched environment under a 12-hour light/dark cycle with food and water available ad libitum.

### In Vivo Control Corneas

Four types of controls were tested. In all of them a similar stromal pocket was dissected, but instead of injecting our biomaterial formulations, they were then respectively processed as follows: (1) Injection of Viscoat (Alcon) in the stromal pocket; (2) Injection of balanced salt solution (BSS) in the stromal pocket; (3) No injection or further treatment; and (4) No injection but photocrosslinking as described above.

### In Vivo Postoperative Follow-Up

After surgery, the animals were followed on a weekly basis for 7 weeks, with slit-lamp examination and wide-angle OCT imaging (Visante and Thorlabs). Examinations were performed under general anesthesia using subcutaneous ketamine (75 mg/kg; Narketan, Vetoquinol, QC, Canada) and medetomidine hydrochloride (0.5 mg/kg; Cepetor, Modern Veterinary Therapeutics, Miami, FL), and atipamezole hydrochloride (Revertor, Modern Veterinary Therapeutics) as reversing agent. Lubricating eye drops (Optive fusion, Allergan, Markan, ON, Canada) were applied repetitively in both eyes during anesthesia. At the end of the study, animals were euthanized with an intraperitoneal dose of pentobarbital sodium (0.5 mL/Kg; Euthanyl, Vetoquinol) injected under general anesthesia and both globes were quickly excised, immerged in 4% paraformaldehyde, and kept at 4° C. overnight.

### Immunohistochemistry

Samples underwent a sucrose gradient from 5% to 20% sucrose (Sigma, S1888-1KG) in 0.1 M TBS and snap-frozen in OCT (FisherSci, 14-373-65). Tissues were sectioned at 10 μm using a cryostat (Leica, CM3050S). Sections were stained with either picrosirius red and alcian blue to visualize collagen and glycosaminoglycans or hematoxylin and eosin. For picrosirius red and alcian blue staining, slides were rinsed in distilled water then incubated in alcian blue (Sigma-Aldrich, A5268-10G) at pH 2.5 for 15 minutes. After a water rinse, samples received picrosirius red solution (Abcam, ab150681) for 45 minutes. Sections were incubated in acidified water for 5 minutes, dehydrated and mounted in Permount mounting media (FisherSci, SP15-100). For hematoxylin and eosin, slides were rinsed in water and immersed into Harris hematoxylin solution (Sigma, HHS32-1L) for 8 minutes. This was followed by a rinse in water, 30 seconds in 1% HCl and 70% ethanol in water, another water rinse, Scott's tap water (0.2% sodium bicarbonate; Sigma, SX0320-1), 1% magnesium sulfate (Sigma, 746452-500G) in distilled water) for 1 minute and finally, another rinse in water. Slides were dipped in 95% ethanol 10 times and incubated for 1 minute in Eosin-phloxine solution (Sigma, HT110316-500ML). Samples were dehydrated and mounted in Permount mounting media (FisherSci, SP15-100). All images were taken on a Zeiss Axio Imager Z2 with an AxioCam MRc color CCD camera (Carl Zeiss, Oberkochen, Germany). Images were processed in FIJI.

### Multivariable Linear Regression Analyses

All analyses were conducted in R (R Core Team (2013). R: A language and environment for statistical computing. R Foundation for Statistical Computing, Vienna, Austria. URL http://www.R-project.org/).

### Example 1—Photoinitiator, Light Dosage, and Ex Vivo Stromal Optimization

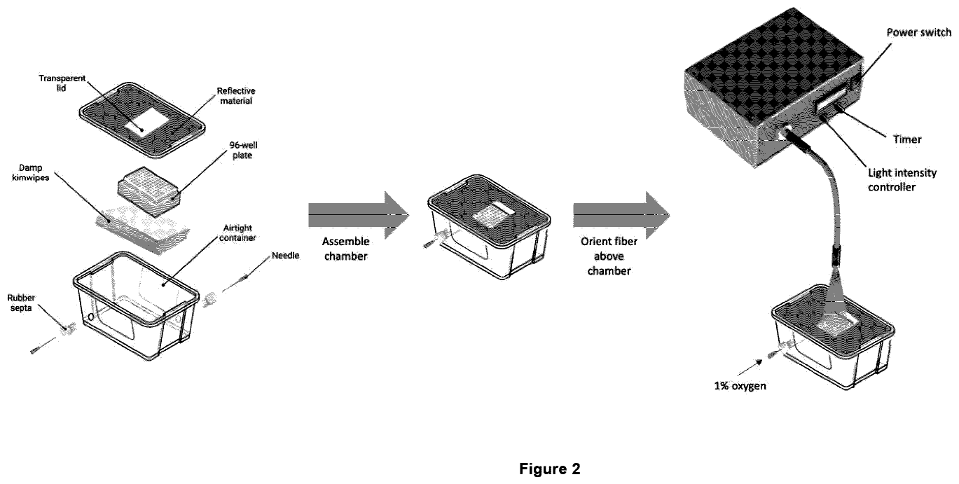

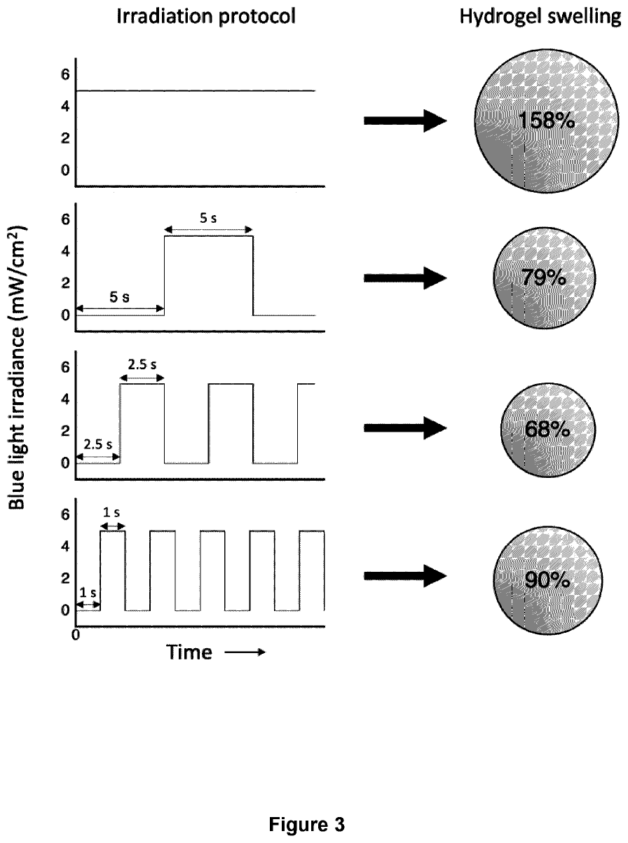

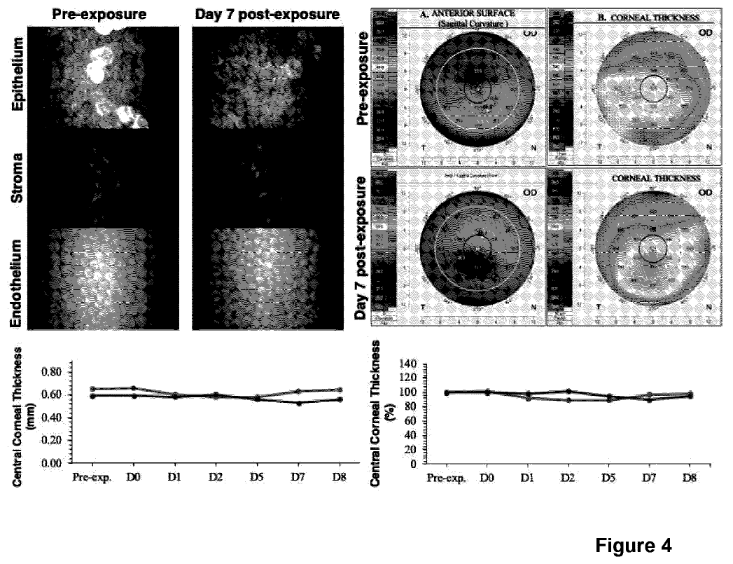

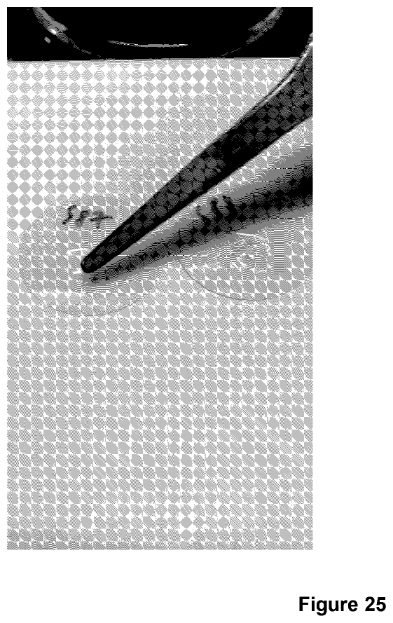

A custom-device for sample irradiation with blue light at 460 nm under low oxygen concentration and high humidity was constructed (FIG. 2). We observed that riboflavin requires ≥1% 02 during irradiation to form hydrogels (FIG. 25). The inventors found that light delivery affected the hydrogel formation. Pulsed irradiation allows for the recovery of the oxygen levels within the hydrogel compared to a continuous dosage of light. FIG. 3 graphically depicts the swelling of a hydrogel produced using different light pulse intervals while keeping the total energy delivered equal in all cases. The optimal condition was found to be light pulsed for 2.5 seconds on and 2.5 seconds off, which was the light cycle chosen for subsequent experiments when the material would be applied into the eye. Excessive blue light irradiation into the eye could damage the retina, something known as blue-light hazard (26). To keep the light dosage under standard safety values (27), the in vivo radiance dosage selected was 835 W/(m²*sr), equivalent to an irradiance of 8.5 mW/cm² for 10 minutes of pulsed light (equivalent to only 5 minutes of light exposure), or a total light dosage of 2.5 J/cm². This radiance is considered under the category of low risk when direct blue light is exposed to the eye for up to 166 minutes (27). In vivo experiments for light toxicity using a feline model demonstrate the safety of the selected light irradiation regime (FIG. 4).

### Example 2—Optimization of the Formulation Composition

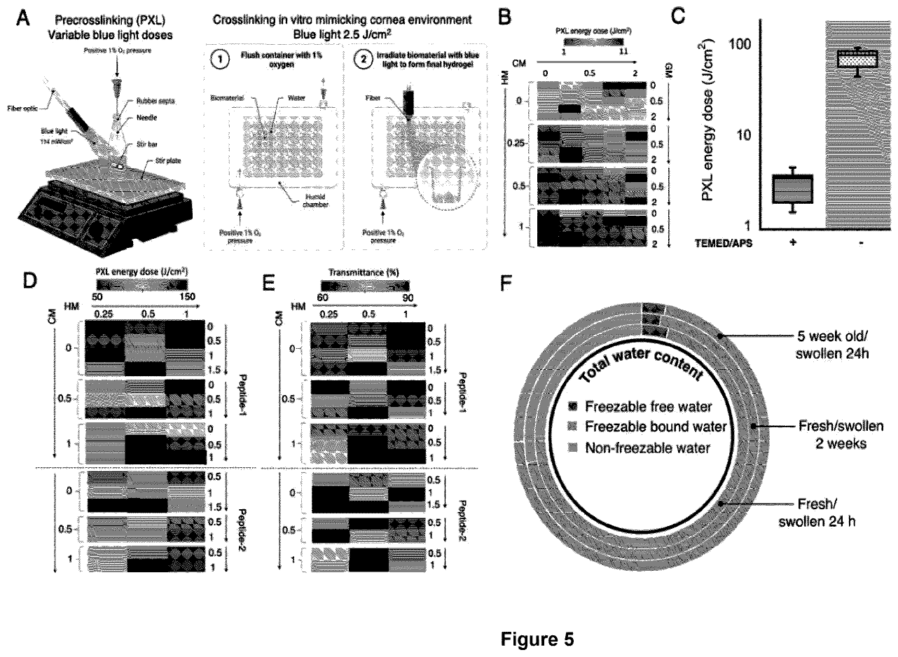

In designing our materials, we started off by using a precrosslink (PXL) step, meaning irradiation before transferring the solution to the syringe for injection, using different light dosages to produce materials with suitable injectability, comparable to that of Viscoat® a clinically used cornea filler (28) (flow index <0.5, Table 1), that can be subsequently crosslinked using 8.5 mW/cm² blue-light for 10 minutes (FIG. 5A).

TABLE 1
Flow behavior index of top hydrogel formulations. Flow behavior
index was taken as the slope of the straight section of
the logarithmic shear rate and shear stress curve.

| | G44 | G50 | G64 | G65 | Viscoat |
|---|---|---|---|---|---|
| Flow | 0.467 | 0.342 | 0.371 | 0.350 | 0.231 |
| behaviour | | | | | |
| index | | | | | |

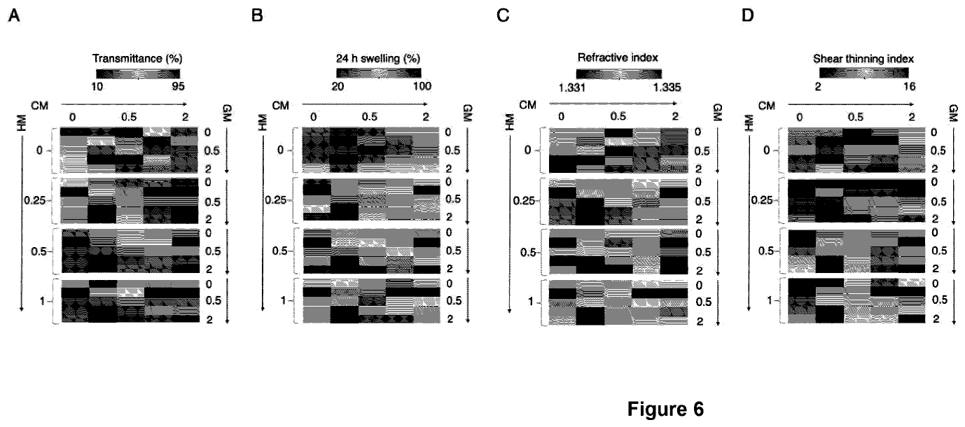

Viscoat is a highly retentive, dispersive viscoelastic solution, composed of 3% sodium hyaluronate, 4% chondroitin sulfate and characterized by low molecular weight, low pseudoplasticity, and low surface tension. This solution is used to fill the anterior chamber to protect the corneal endothelium during cataract surgery (29). As a roadmap to develop materials that would not need to use gelatin or collagen derivatives, the inventors first tested formulations with varying concentrations of hyaluronic acid methacrylate (HM), chondroitin methacrylate (CM), and gelatin methacrylate (GM) at different light intensities (FIG. 1). Associations between the composition mixture (HM, CM, and GM) and PXL energy dose, swelling, transparency, shear-thinning index, and refractive index are depicted in FIG. 6 whereas Table 2 below shows the complete multivariable linear regression analyses.

TABLE 2
Multivariable linear regression estimates of the associations
between polymer concentration and various hydrogel properties
(energy dose, shear thinning index, 24 h swelling and transmittance).
Beta coefficients and corresponding p-values.

| | Energy dose | | Shear thinning | | 24 h swelling | | | |
|---|---|---|---|---|---|---|---|---|
| Independent | (J/cm²) | | index | | (%) | | Transmittance | |
| variable | β(SE) | p-value | β(SE) | p-value | β(SE) | p-value | β(SE) | p-value |
| GM | −0.57 | 0.017 | 0.52 | 0.30 | 12.96 | <0.0001 | −12.97 | <0.0001 |
| | (0.23) | | (0.51) | | (2.16) | | (2.29) | |
| CM | 0.68 | 0.005 | 0.33 | 0.66 | 3.13 | 0.19 | −11.05 | <0.0001 |
| | (0.24) | | (0.49) | | (2.40) | | (2.34) | |
| HM | −2.94 | <0.0001 | 4.16 | <0.0001 | 27.18 | <0.0001 | −10.99 | 0.02 |
| | (0.33) | | (0.79) | | (4.14) | | (4.74) | |

β = regression coefficient;
GM = gelatin methacrylate;
CM = chondroitin methacrylate;
HM = hyaluronic methacrylate

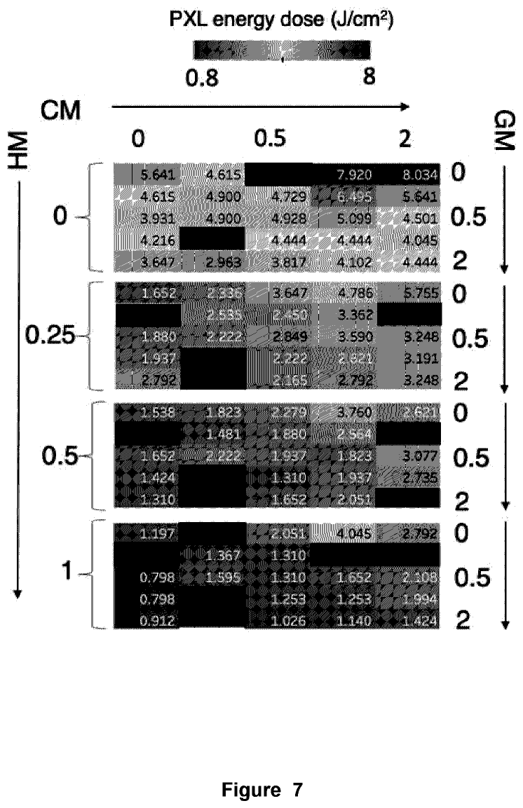

In selecting the optimal concentrations in the formulation, we considered those materials with lower energy dose (<5 J/cm²), lower swelling (<60%), higher transparency (>55%), high shear thinning index (>10), and refractive indexes similar to that of vitreous humor (1.336) (30). Formulations with high concentrations of HM needed less energy to PXL compared to those without HM. In contrast, formulations with high concentrations of CM needed more energy to PXL. Adding GM resulted in lower energies but to a lesser extent than HM (see FIG. 5B and FIG. 7 for a qualitative and quantitative representation of PXL energy values, respectively).

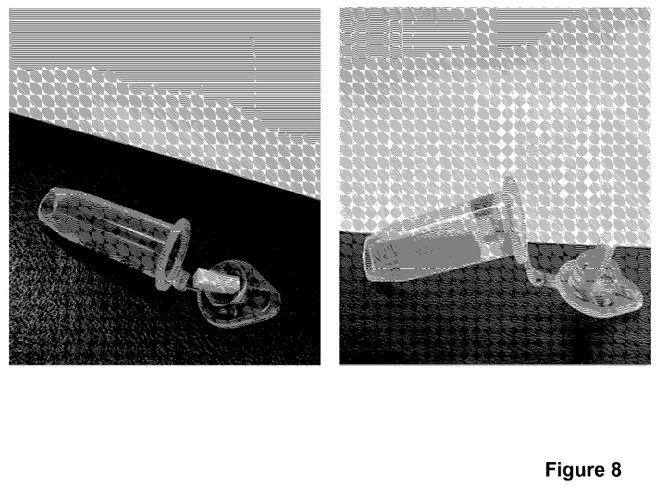

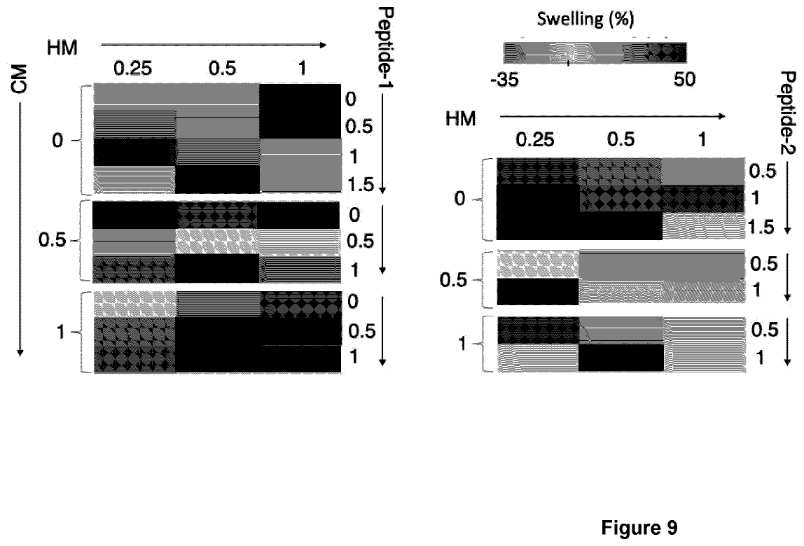

The transition from formulations crosslinked with ammonium persulfate (APS) and riboflavin to solely riboflavin also led to slower crosslinking rates. The APS/tetramethylethylenediamine (TEMED) system initially employed to help facilitate precrosslinking led to short gelation times (<3 min; FIG. 8). However, when APS/TEMED was removed from the formulations to minimize toxicity and corneal edema (31, 32). This also resulted in the energy dose required for each formulation during precrosslinking to increase (p<0.001; FIG. 5C). The PXL solutions needed an energy dose between 48-151 J/cm² compared to an energy dose between 0.8-8 J/cm² for the APS/TEMED containing formulations (FIG. 5C). In further exploring the development of our peptide-containing formulations, we pre-crosslinked the materials to allow for a reduced final in situ crosslinking energy dose of 2.5 J/cm². By doing this, we overcome the limitation of using high light dosages to obtain a material capable of in situ crosslinking in the cornea without producing light-mediated damage to the eye. After replacing gelatin methacrylate for our custom-made peptides in the formulations, the energy required to reach each biomaterial's PXL point significantly increased (FIG. 5D). The influence of peptide concentration on hydrogel swelling showed that replacing GM by peptide-1 did affect swelling (FIG. 9, p<0.05), whereas peptide-2 concentration did not have a significant effect on swelling (FIG. 9). Swelling of peptide-based materials was carried out in saline solution with 5% dextran, which has an osmotic pressure near the swelling pressure of the corneal stroma (33). From the initial set of materials, only eleven had swelling within ±10% and moved forward for further testing. The materials prepared using peptides instead of gelatin remained transparent (>60% transmittance vs. human cornea transparency 55-95%, FIG. 5E) (34-36), and had high water content, remaining stable up to 5-weeks post-preparation of the PXL formulation (see representative example FIG. 5F).

The top hydrogel formulations underwent DSC testing after swelling in saline containing 5% wt/wt dextran for 24 h and 2 weeks, sequentially. DSC analysis is often performed on hydrogels to study the three different hydrogel states of water: non-freezing bound water, freezing bound water, and freezing free water. The non-freezing and freezing bound water form the primary and secondary hydration shell of the hydrophilic polymer network, respectively, whereas the free water does not interact with the polymer structure. The freezable water content (W_{f}) was calculated from the enthalpy of melting water in the hydrogel (area under the endothermic peak around 0° C.) (ΔH_{endo}) and the enthalpy of melting of pure water (334 J/g) (ΔH_{w}) using the following equation:

$${W}_{f} = \frac{Δ {H}_{endo}}{Δ {H}_{w}} \times 100 \%$$

The fraction of freezable water that is bound to the polymer network (W_{fb}) and the fraction of freezable water that can flow freely in the hydrogel (W_{ff}) were determined using the subsequent equations:

$${W}_{fb} = \frac{Δ {H}_{exo}}{Δ {H}_{w}} \times 100 \%$$

$${W}_{ff} = {W}_{f} - {W}_{fb}$$

Where ΔHₑₓₒ is the enthalpy of crystallization of water in the hydrogel (area under the exothermic peak below −10° C. The non-freezing bound water content was then taken as the difference between the equilibrium water content and the freezing water content. This method used to establish the percentage of freezable bound and unbound water in hydrogels assumes that the enthalpy of melting of freezable bound water is equivalent to free water.

### Total Energy Dose

The total amount of blue light energy delivered per square centimeter or fluence (F) was determined using the following equation:

$$F = {E}_{e} \cdot t \cdot v$$

where Eₑ, t, and v are irradiance, exposure time, and pulse frequency, respectively. For in situ blue light crosslinking, the irradiance, exposure time and pulse frequency were 8.5 mW/cm², 600 s, and 0.5 (2.5 s blue light for every 5 s), respectively. As a result, the total energy dosage of blue light in situ was ~2500 mJ/cm² or 2.5 J/cm².

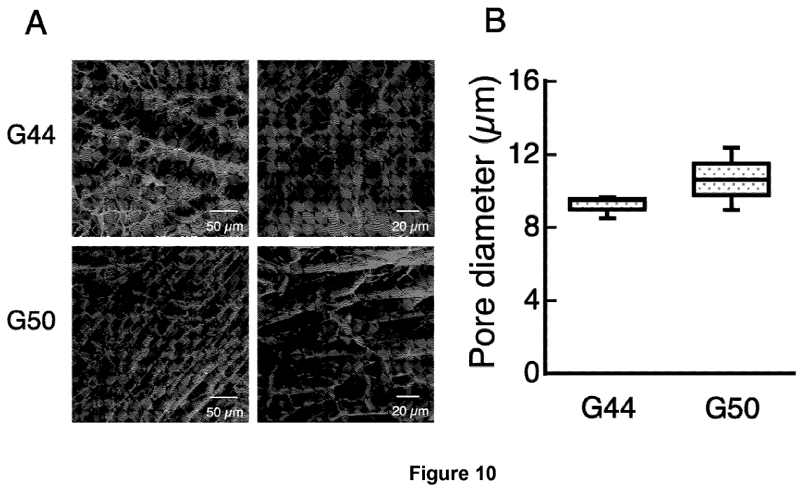

The top four hydrogels that demonstrated good stability after 2 weeks of swelling, as shown in the differential scanning calorimetry (DSC) results detailed in Table 3, were further characterized. Hydrogels exhibited a glass transition temperature between 60-66° C., similar to the denaturation temperature of a biomimetic cornea and native cornea (Table 3). The top hydrogel candidates displayed slower degradation kinetics in 5 U/mL collagenase (G44 0.057±0.006 mg/h and G50 0.055±0.001 mg/h) compared to biomimetic cornea control (0.33±0.0045 mg/h). Pore sizes as measured using low temperature cryo-SEM indicated that the materials' pore size values were in the range of 9-11 μm (FIG. 10).

TABLE 3
Differential scanning calorimetry Tg temperature. Hydrogel samples
between 6-13 mg were placed in Tzero hermetic pans, sealed, and
underwent DSC protocol where hydrogels were cooled to 10°
C. and heated to 90° C. at a heating/cooling rate of 10° C./min.

| Sample | Tg (° C.) |
|---|---|
| G44 | 65 ± 11.0 |
| G50 | 61 ± 3.9 |
| Biosynthetic cornea | 61 ± 1.9 |

### Example 3—Ex Vivo and In Vitro Testing

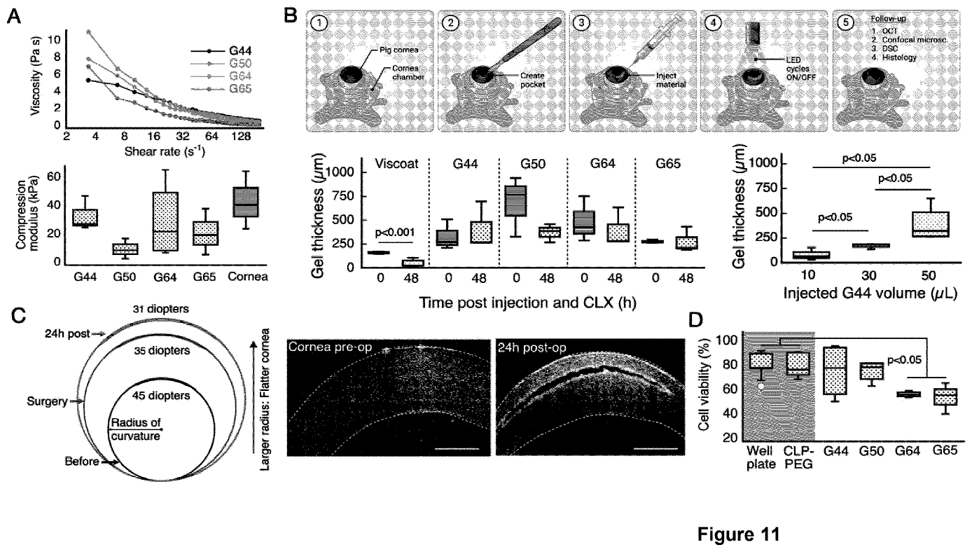

All four selected formulations showed viscosity comparable to that of Viscoat (FIG. 11A top). Further, fully crosslinked materials identified as G44, G50, G64, and G65 displayed compression moduli that were not significantly different from that obtained for pig corneas (p>0.05, FIG. 11A bottom). The compositions identified as G44, G50, G64, and G65 are detailed in Table 4 below.

TABLE 4
Composition of top hydrogel formulations. Hydrogels
were also composed of 9 wt % 8-arm PEG acrylate,
3.4 wt % PEGDA (Mn 700 g/mol), 0.7 wt % PEGDA (Mn
250 g/mol) 10 mM HEPES, and 0.14 mM riboflavin.

| | Peptide 1 | Peptide 2 | CM | HM |
|---|---|---|---|---|
| | (%) | (%) | (%) | (%) |
| G44 | 0.5 | 0 | 0.5 | 1.0 |
| G50 | 0 | 1.5 | 0 | 1.0 |
| G64 | 0 | 1.0 | 1.0 | 0.25 |
| G65 | 0 | 1.0 | 0.5 | 0.5 |

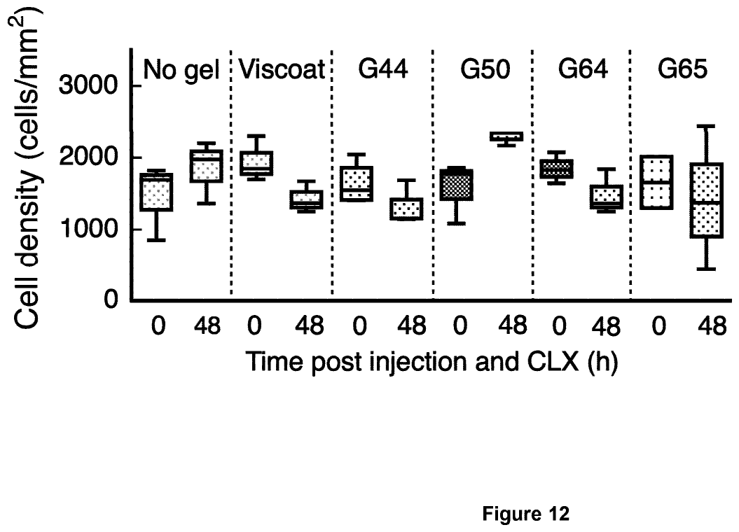

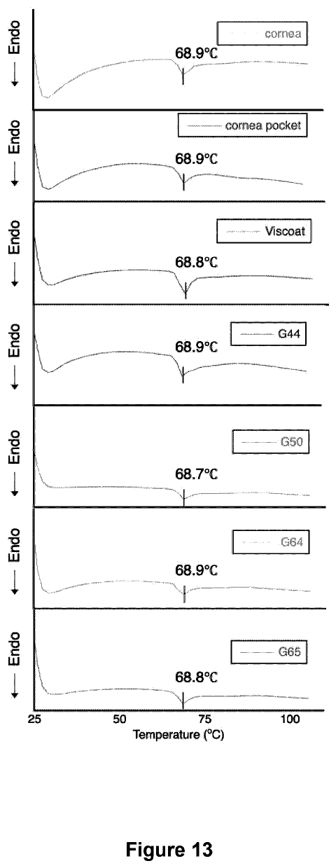

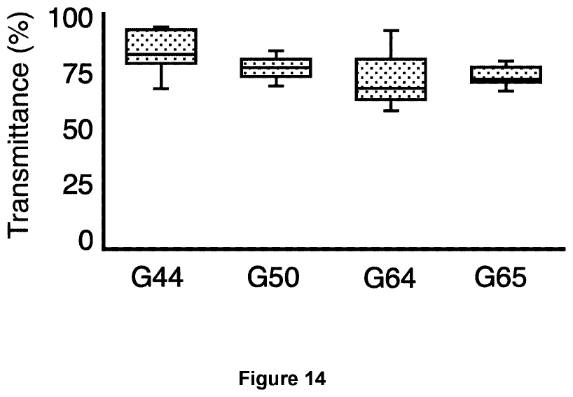

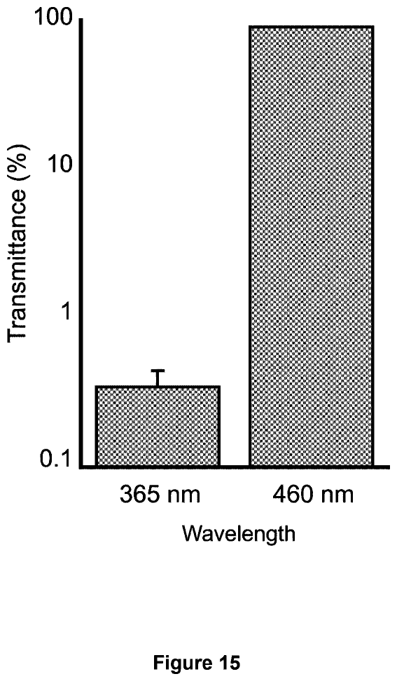

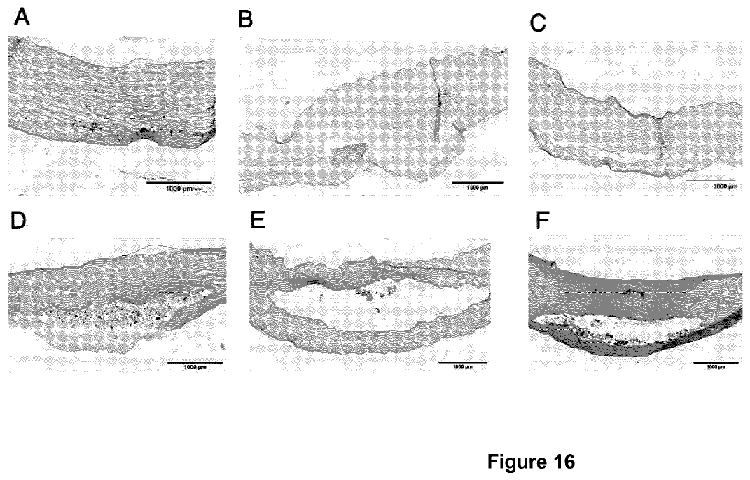

Ex vivo intrastromal injections in pig corneas (FIG. 11B top for a schematic) showed that fully crosslinked formulations remained stable for 48 h after injection as documented by optical coherence tomography (OCT) imaging (FIG. 11B bottom left). Cornea stromal cell structure was not adversely affected by the creation of the intrastromal pocket nor by the presence of the injected materials over the 48 h period based on Confoscan imaging (FIG. 12). DSC results confirmed the injected material did not significantly affect the cornea denaturation temperature (FIG. 13). Further, cornea transparency was not significantly impacted by the presence of any of the four injected materials (FIG. 14). Porcine cornea thickness could be controlled by injecting different volumes of photoactivated material into the stromal pocket (FIG. 11B Bottom right). Cornea curvature was altered significantly when keratoconic lenses (Centracone and RGP) were used during the crosslinking process (FIG. 11C). The keratoconic lenses were transparent to blue light allowing for photocrosslinking to be completed as normal without needing to increase the dosage of blue light (FIG. 15). H&E-stained pig cornea sections showed differential degrees of incorporation of the hydrogels into the pig cornea (FIG. 16). Picrosiruis red collagen staining and alcian blue glycosaminoglycans staining showed that G44 was intercalated into the stromal lamellae, giving the observed green appearance. The G50, G64, and G65 hydrogels showed red collagen staining like the untreated control cornea, indicative of a firmer hydrogel that remained at the injection site.

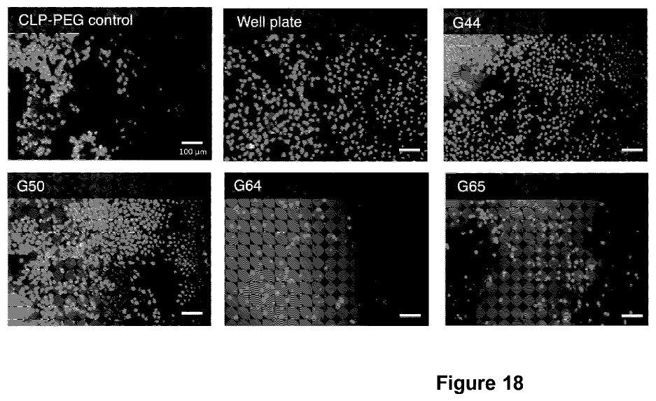

Immortalized human corneal epithelial cells attached and grew on fully crosslinked materials with percentages of viability like those observed in the control groups (FIG. 11D and FIG. 18). Formulations G44 and G50 displayed numbers of viable cells comparable to those of a non-photocrosslinked CLP-PEG hydrogel control, crosslinked by DMTMM, that has successfully promoted regeneration in mini-pigs (p>0.05) (37). However, G64 and G65 showed a statistically significant reduction in the number of viable cells (p<0.001; see FIG. 11D) compared to the CLP-PEG control. Further, ex vivo injection of the G44 and G50 formulations in excised pig corneas showed that these formulations are capable of reshaping corneal curvature, see FIG. 11C. Thus, the inventors have centered the discussion on the in vivo results obtained using G44 and G50 formulations in present rodent model.

### Example 4—In Vivo Assessment in a Rat Model

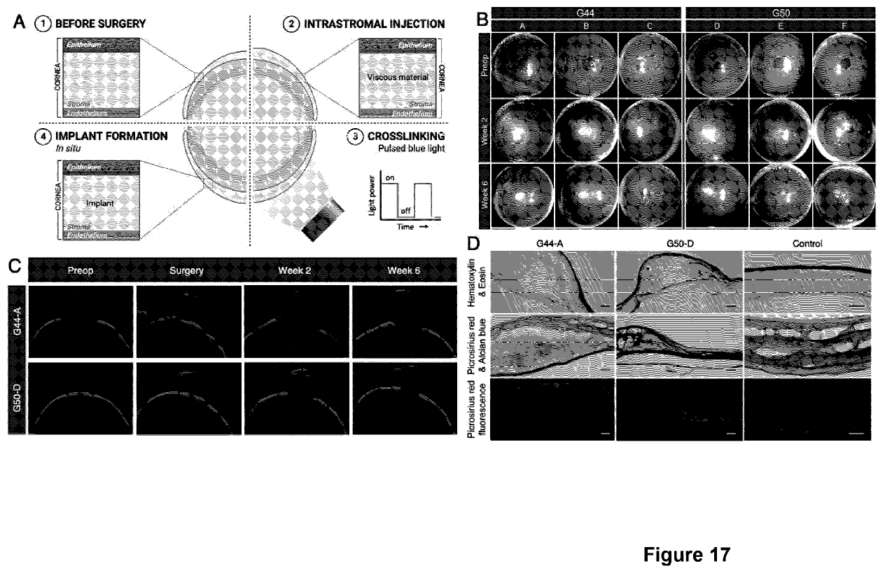

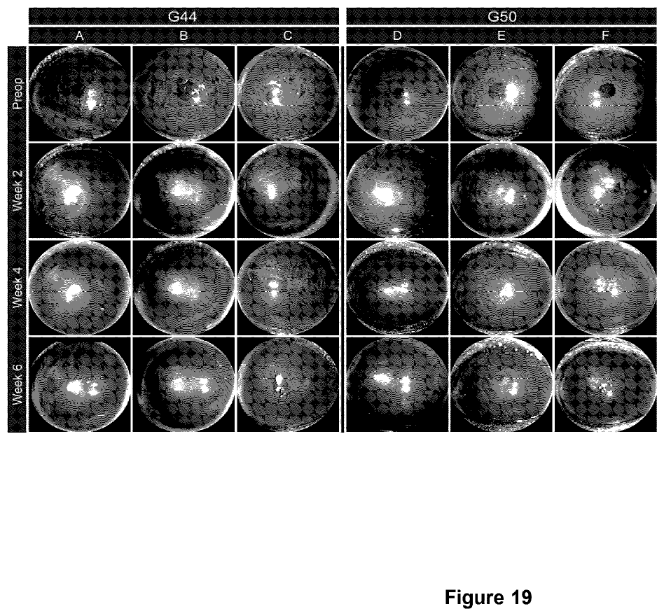

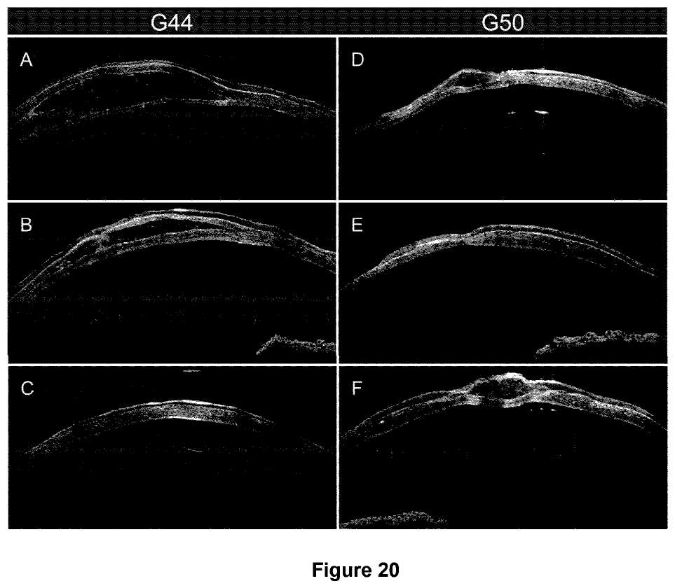

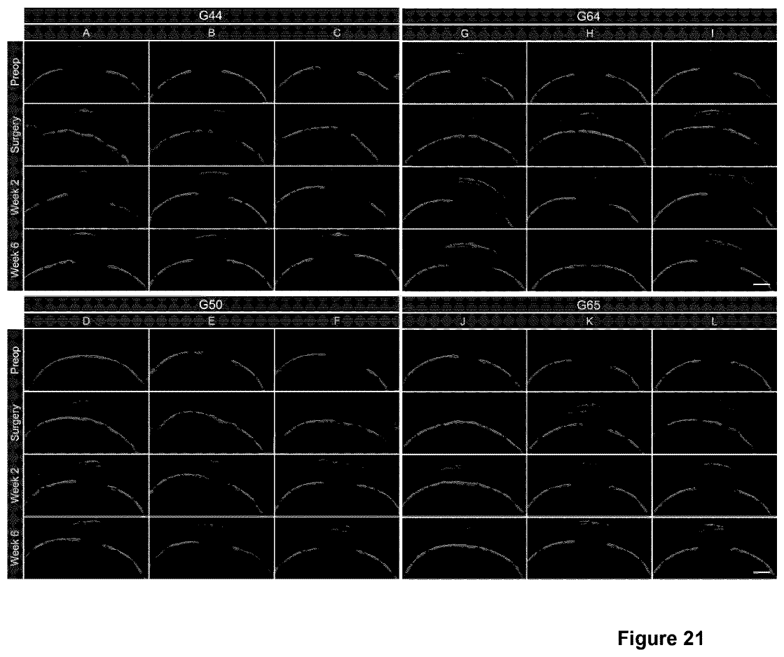

A schematic representation for the in vivo experimental design is presented in FIG. 17. Evolution of the intracorneal injections of the four formulations is illustrated in FIG. 17B and FIG. 19 for the slit lamp photos and representative Visante OCT images. The higher resolution Thorlabs OCT imaging performed at the end of the study is detailed in FIG. 20. Formulations were tested in triplicate (G44 in animals A, B, and C; and G50 in D, E, and F). Visante OCT imaging throughout the study period for all groups are depicted in FIG. 21.

In cornea G44-A, significantly more material was injected compared to the other corneas (FIG. 17C). The central thickness of this implant was 470 μm on OCT cross-section immediately after crosslinking in a cornea had a normal thickness of 210 μm at the same location prior to surgery. The injected volume decreased by approximately half during the first 24 h after surgery and down to 160 μm after 2 weeks, remaining stable (160 μm) afterward until the end of the study (FIG. 20).

For one cornea from each group (G44-C and G50-E), the injected implant was rapidly lost after surgery and no hydrogel was detectable by OCT at 6 weeks post-operation (FIG. 21). In the other samples, the shape changes were stable, with implant thicknesses at Day1/Week6 of G44-B: 130/120 μm, G50-D: 120/120 μm, and G50-F: 120/130 μm, respectively. In all corneas but G44-C and G50-E, a mild thickening of the epithelium was observed in the area surrounding the implants (FIG. 21), most probably due to the chronic change in corneal shape and surface curvature induced by the implants, a finding that was not observed in G44-C and G50-E, the two treated corneas without an implant.

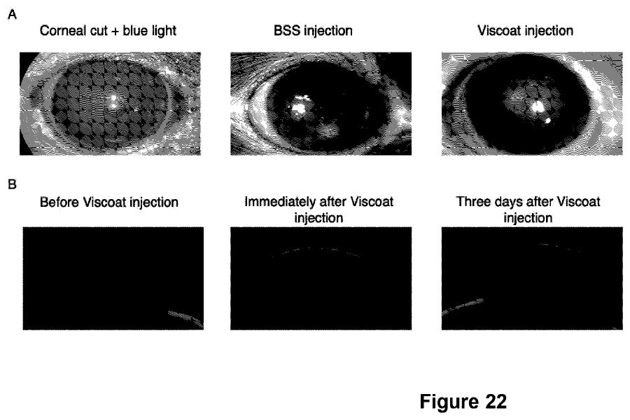

The G44 or G50 formulations did not appear to induce inflammation, none of the injected corneas developed visible neovascularization, and all corneas healed with minimal scarring, which indicates that the formulations were biocompatible and non-cytotoxic. Results obtained with the surgical control corneas injected with Balanced Salt Solution (BSS), a solution used in eye surgery for surface and intraocular irrigation, or Viscoat, a viscoelastic material used in cataract surgery to fill the anterior chamber and to protect the cornea, are shown in FIG. 22. The Viscoat vanished during the first few days after surgery, while the BSS instantaneously leaked outside the wound. All control corneas showed some degree of scarring, whether Viscoat or BSS was injected or not, and with or without irradiation with blue light, which confirmed the tendency of rat corneas to generate scar tissue in response to surgical wounds. The degree of corneal scarring observed in controls was overall like that obtained after injection of G44 or G50 formulations (see FIG. 19), which also indicates a low degree of toxicity for these formulations, if any.

H&E sections through the corneas of treated animals clearly showed the presence of the hydrogel in the stroma of two implants (G44-A and G50-D; FIG. 17D). Picrosirius red and alcian blue staining showed that the injected hydrogel, visualized by the bluish-purple staining of mainly the hyaluronic acid and chondroitin sulphate, was incorporated between the collagen lamellae of the cornea in the G50-D treated cornea. Apart from the shape change, the overlying epithelium, stroma, and innermost endothelial layer remained healthy. Epithelial thickening around the implanted areas and in some areas epithelial smoothing of irregular surfaces was also observed. No infiltrates of immune cells or signs of cell death were observed and there was no corneal neovascularization.

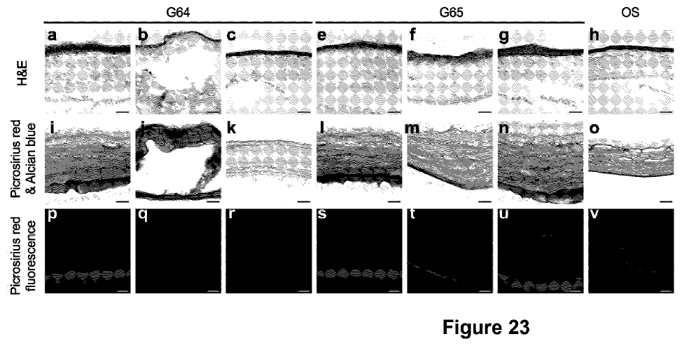

Histology results for corneas from Groups G64 and G65 that did not perform as well as G44 and G50 in vitro are shown in FIG. 23. In Group G65, there was picrosirius red-alcian blue staining under the corneal endothelium of the injected corneas but not in the contralateral untreated controls suggesting that the biomaterials could have flowed out of the corneas and collected at the interface between the endothelium and aqueous humor.

Corneal thinning is a significant problem for which there are not effective solutions. Corneal crosslinking only serves to stabilize already thinning or thinned corneas but does not replace the largely lost collagenous extracellular matrix. In this study, we developed light-activated injectable biomaterials with a range of properties that could be fine-tuned to resemble the human cornea by adjusting the concentration of biopolymers and peptides in the hydrogel formulations. In the two formulations that we selected for resembling native corneal properties, we were able to achieve comparable compressive moduli to those of excised pig corneas, showing their potential as bulking agents for thinning corneas.

### Chemical Precrosslinking

Corneal crosslinking with Riboflavin and UVA is an FDA-approved therapy to increase anterior cornea stiffness (38). However, limited applications for intrastromal therapies using riboflavin have been explored mostly due to the low penetration of UVA light (39). Riboflavin does absorb in the 400-460 nm region and has a dual behaviour as a photosensitizer, which allows it to work under low oxygen concentrations (40, 41). Corneal crosslinking with riboflavin has been described to be affected by the oxygen concentration in the stroma and its performance being reduced when oxygen is completely depleted, which happens just in a few seconds (42). Further, any intrastromal application must account for a highly wet environment (80% of the stroma is water) (43).

The precrosslinking step significantly increased the viscosity of the material until its viscosity was comparable to Viscoat®, allowing the material to be injected into the stroma without leaking from the intrastromal pocket before final crosslinking. Overall, biomaterial intrastromal injection remained a smooth and easy process for clinical application by ophthalmologists. Our results demonstrate that using a precrosslinking step allows for reducing the amount of light required for forming a stable hydrogel in situ. This represents an upgrade over other reported photoactivated materials developed for cornea repair, all of which require energy levels between ≈2 to 30 times higher than our material's energy requirement for in vivo crosslinking (44-47). In clinical conditions, reduction of the amount of light delivered to the eye would translate into a faster and safer procedure (less than 5-10 minutes of in situ crosslinking). A quicker treatment would be easier for the patient under topical anesthesia. It would also shorten the period during which eye movement should be minimized to ensure stability of the injected biomaterial volume and shape until it turns into a soft hydrogel. A good control of the corneal front surface curvature is of primary importance for adequate refraction of the light within the eye, the other most important parameter governing retinal image quality being transparency (70-90% in the visible range).

### Toxicity Testing and Biocompatibility

The materials and their processing were screened for their absence of toxicity. In culture, corneal epithelial cells attachment, growth, and viability over G44 and G50 hydrogels were like those of the LiQD Cornea controls, which confirmed their good cytocompatibility. On the other hand, some of the other photoactivated materials reported in the literature used compounds that can be toxic to human tissues such as triethanolamine (17, 48, 49). Furthermore, the low energy blue light illumination used for photocrosslinking improves light penetration and thus reduces the risk of phototoxicity compared to UVA currently used in the clinic for the crosslinking of pathologically thin corneas.

### Full Crosslinking and Shape Control

Our technology uses seconds-scale pulsed light instead of continuous light, which allowed for in situ oxygen replenishing within the material during photocrosslinking and thus contributed to ensure full gelation of the intrastromal implant. In the interest of future clinical applications, ex vivo and in vivo short-term testing showed that the designed peptide-based materials can be injected and maintain shape in large, compact, very thick, and flat corneas such as that of the pig (with a diameter of 14.0 to 16.6 mm, a thickness of 666 μm [543 μm to 797 μm] in vivo, increasing to >1000 μm ex vivo, and an anterior curvature of 40 Diopters [36.5 to 41 D]) (50-53), as well as in very small, thin, flimsy and much steeper corneas such as that of the rat (central thickness: 156-170 μm; anterior radius of curvature 3.06 mm) (54, 55). Further, ex vivo testing in pig corneas demonstrated that implant thickness could be adjusted by varying the quantity of material injected into the stromal pocket. In addition to cornea thickness tunability, cornea curvature can also be modified by photocrosslinking the injected material using stiff lenses which maintain the desired shape until the material is fully crosslinked. As a result, cornea steepening which is a major problem associated with keratoconus may be corrected by placing keratoconus lenses during the light irradiation step. In both types of corneas, injected materials were able to withstand the intracameral pressure until crosslinking was over, even in the presence of an open stromal wound that would have been responsible for complete leakage if the biomaterial had been more liquid. Balanced salt solution for instance, which was injected in control corneas for comparison purposes, was instantaneously eliminated through the wound. While a shrinkage factor is to be considered, as G44 and G50 implants decreased in size by 53% and 58%, respectively, during the first 3 weeks after implantation and crosslinking, the implants remained stable in size and volume afterwards.

### Potential Therapeutic Applications

Our results suggest that these materials could be used for the treatment of a large spectrum of eye diseases characterized by a lack of corneal tissue, including various types of post-traumatic corneal tissue loss, or the corneal thinning seen in conditions, for example, but not limited to, keratoconus or pellucid degeneration, and in some of the refractive errors of the eye.

All references cited herein are incorporated by reference.

### REFERENCES

1. H. Aghamohammadzadeh, R. H. Newton, K. M. Meek, X-ray scattering used to map the preferred collagen orientation in the human cornea and limbus. Structure 12, 249-256 (2004).
2. A. P. Marques et al., Global economic productivity losses from vision impairment and blindness. eClinicalMedicine 35, 100852 (2021).
3. S. R. Flaxman et al., Global causes of blindness and distance vision impairment 1990-2020: a systematic review and meta-analysis. Lancet Glob Health 5, e1221-e1234 (2017).
4. Y. S. Rabinowitz, Keratoconus. Surv Ophthalmol 42, 297-319 (1998).
5. H. Hashemi et al., The Prevalence and Risk Factors for Keratoconus: A Systematic Review and Meta-Analysis. Cornea 39, 263-270 (2020).
6. A. Gordon-Shaag, M. Millodot, E. Shneor, Y. Liu, The Genetic and Environmental Factors for Keratoconus. BioMed Res Int 2015, 795738 (2015).
7. Z. Mackiewicz et al., Collagenolytic proteinases in keratoconus. Cornea 25, 603-610 (2006).
8. C. Chan, Corneal Cross-Linking for Keratoconus: Current Knowledge and Practice and Future Trends. Asia Pac J Ophthalmol (Phila) 9, 557-564 (2020).
9. A. S. McCall et al., Mechanisms of corneal tissue cross-linking in response to treatment with topical riboflavin and long-wavelength ultraviolet radiation (UVA). Invest Ophthalmol Vis Sci 51, 129-138 (2010).
10. S. K. Subasinghe, K. C. Ogbuehi, G. J. Dias, Current perspectives on corneal collagen crosslinking (CXL). Graefes Arch Clin Exp Ophthalmol 256, 1363-1384 (2018).
11. F. Raiskup, E. Spoerl, Corneal crosslinking with riboflavin and ultraviolet A. I. Principles. Ocul Surf 11, 65-74 (2013).
12. P. Gain et al., Global Survey of Corneal Transplantation and Eye Banking. JAMA Ophthalmol 134, 167-173 (2016).
13. P. Fagerholm et al., A biosynthetic alternative to human donor tissue for inducing corneal regeneration: 24-month follow-up of a phase 1 clinical study. Sci Transl Med 2, 46ra61 (2010).
14. M. M. Islam et al., Biomaterials-enabled cornea regeneration in patients at high risk for rejection of donor tissue transplantation. NPJ Regen Med 3, 2 (2018).
15. C. D. McTiernan et al., LiQD Cornea: Pro-regeneration collagen mimetics as patches and alternatives to corneal transplantation. Sci Adv 6, eaba2187 (2020).
16. S. Chen, M. J. Mienaltowski, D. E. Birk, Regulation of corneal stroma extracellular matrix assembly. Exp Eye Res 133, 69-80 (2015).
17. E. Shirzaei Sani et al., Sutureless repair of corneal injuries using naturally derived bioadhesive hydrogels. Sci Adv 5, eaav1281 (2019).
18. J. M. Kelso, The gelatin story. J Allergy Clin Immunol 103, 200-202 (1999).
19. NTP toxicology and carcinogenesis studies of triethanolamine (Cas No. 102-71-6) in B6C3F1 mice (dermal studies). Natl Toxicol Program Tech Rep Ser, 5-163 (2004).
20. P. N. Youssef, N. Sheibani, D. M. Albert, Retinal light toxicity. Eye (Lond) 25, 1-14 (2011).
21. E. Spoerl, A. Hoyer, L. E. Pillunat, F. Raiskup, Corneal cross-linking and safety issues. Open Ophthalmol J 5, 14-16 (2011).
22. D. Z. Reinstein, M. Gobbe, T. J. Archer, R. H. Silverman, D. J. Coleman, Epithelial, stromal, and total corneal thickness in keratoconus: three-dimensional display with artemis very-high frequency digital ultrasound. J Refract Surg. 26, 259-271 (2010).
23. H. P. Iseli, E. Spoerl, P. Wiedemann, R. R. Krueger, T. Seiler, Efficacy and safety of blue-light scleral cross-linking. J Refract Surg 24, S752-755 (2008).
24. A. R. Irizarry Rovira et al., Scientific and Regulatory Policy Committee Points to Consider: Histopathologic Evaluation in Safety Assessment Studies for PEGylated Pharmaceutical Products. Toxicol Pathol 46, 616-635 (2018).
25. G. Huerta-Ángeles, K. Nešporová, G. Ambrožová, L. Kubala, V. Velebný, An Effective Translation: The Development of Hyaluronan-Based Medical Products From the Physicochemical, and Preclinical Aspects. Front Bioeng Biotechnol 6, 62 (2018).
26. J. D. Bullough, The Blue-Light Hazard: A Review. J Illuminating Eng Soc 29, 6-14 (2000).
27. E. L. C. Federation, in Data on case B: Looking at a light source (radiance). (https://moodle.polymtl.ca/pluginfile.php/354010/mod resource/content/2/CELMA-ELC_LED_WG%28SM%29011_ELC_CELMA_position_paper_optical_safety_LED_lighting_Final 1st_Edition_July2011.pdf, 2011).
28. N. Maár, A. Graebe, G. Schild, M. Stur, M. Amon, Influence of viscoelastic substances used in cataract surgery on corneal metabolism and endothelial morphology: comparison of Healon and Viscoat. J Cataract Refract Surg 27, 1756-1761 (2001).
29. G. Rainer et al., Natural course of intraocular pressure after cataract surgery with sodium chondroitin sulfate 4%-sodium hyaluronate 3% (Viscoat). Ophthalmology 112, 1714-1718 (2005).
30. Y. C. Chang et al., In vivo measurement of the human crystalline lens equivalent refractive index using extended-depth OCT. Biomed Opt Express 10, 411-422 (2019).
31. S. Duan, W. Zhu, L. Yu, J. Ding, Negative cooperative effect of cytotoxicity of a di-component initiating system for a novel injectable tissue engineering hydrogel. Chinese Sci Bull 50, 1093-1096 (2005).
32. W. N. Albrecht, R. L. Stephenson, Health hazards of tertiary amine catalysts. Scand J Work Environ Health 14, 209-219 (1988).
33. C. H. Dohlman, B. O. Hedbys, S. Mishima, The swelling pressure of the corneal stroma. Invest Ophthalmol 1, 158-162 (1962).
34. J. D. Mallet, P. J. Rochette, Wavelength-dependent ultraviolet induction of cyclobutane pyrimidine dimers in the human cornea. Photochem Photobiol Sci 12, 1310-1318 (2013).
35. T. J. Freegard, The physical basis of transparency of the normal cornea. Eye (Lond) 11 (Pt 4), 465-471 (1997).
36. K. M. Meek, C. Knupp, Corneal structure and transparency. Prog Retin Eye Res 49, 1-16 (2015).
37. F. C. Simpson et al., Collagen analogs with phosphorylcholine are inflammation-suppressing scaffolds for corneal regeneration from alkali burns in mini-pigs. Commun Biol 4, 608 (2021).
38. D. Wu et al., Corneal Cross-Linking: The Evolution of Treatment for Corneal Diseases. Front Pharmacol 12, 686630 (2021).
39. L. Kolozsvari, A. Nogradi, B. Hopp, Z. Bor, UV absorbance of the human cornea in the 240- to 400-nm range. Invest Ophthalmol Vis Sci 43, 2165-2168 (2002).
40. M. Munoz et al., Riboflavin Surface Modification of Poly(vinyl chloride) for Light-Triggered Control of Bacterial Biofilm and Virus Inactivation. ACS Appl Mater Interfaces 13, 32251-32262 (2021).
41. M. A. Munoz et al., Different cell death mechanisms are induced by a hydrophobic flavin in human tumor cells after visible light irradiation. J Photochem Photobiol B 103, 57-67 (2011).
42. P. Kamaev, M. D. Friedman, E. Sherr, D. Muller, Photochemical kinetics of corneal cross-linking with riboflavin. Invest Ophthalmol Vis Sci 53, 2360-2367 (2012).
43. Z. D. Taylor et al., THz and mm-Wave Sensing of Corneal Tissue Water Content: Electromagnetic Modeling and Analysis. IEEE Trans Terahertz Sci Technol 5, 170-183 (2015).
44. A. T. Hillel et al., Photoactivated composite biomaterial for soft tissue restoration in rodents and in humans. Sci Transl Med 3, 93ra67 (2011).
45. K. Hosoyama et al., Nanoengineered Electroconductive Collagen-Based Cardiac Patch for Infarcted Myocardium Repair. ACS Appl Mater Interfaces 10, 44668-44677 (2018).
46. Y. Wang, I. E. Kochevar, R. W. Redmond, M. Yao, A light-activated method for repair of corneal surface defects. Lasers Surg Med 43, 481-489 (2011).
47. S. Sharifi et al., Tuning gelatin-based hydrogel towards bioadhesive ocular tissue engineering applications. Bioact Mater 6, 3947-3961 (2021).
48. C. S. Bahney et al., Visible light photoinitiation of mesenchymal stem cell-laden bioresponsive hydrogels. Eur Cell Mater 22, 43-55; discussion 55 (2011).
49. L. Degoricija, C. S. Johnson, M. Wathier, T. Kim, M. W. Grinstaff, Photo cross-linkable Biodendrimers as ophthalmic adhesives for central lacerations and penetrating keratoplasties. Invest Ophthalmol Vis Sci 48, 2037-2042 (2007).
50. J. Heichel, F. Wilhelm, K. S. Kunert, T. Hammer, Topographic Findings of the Porcine Cornea. Med Hypothesis Discov Innov Ophthalmol 5, 125-131 (2016).
51. L. Jay et al., Determination of porcine corneal layers with high spatial resolution by simultaneous second and third harmonic generation microscopy. Opt Express 16, 16284-16293 (2008).
52. C. Faber, E. Scherfig, J. Prause, K. Sorensen, Corneal Thickness in Pigs Measured by Ultrasound Pachymetry In Vivo. J Lab Anim Sci 35, (2008).
53. I. Sanchez, R. Martin, F. Ussa, I. Fernandez-Bueno, The parameters of the porcine eyeball. Graefes Arch Clin Exp Ophthalmol 249, 475-482 (2011).
54. A. Chaudhuri, P. E. Hallett, J. A. Parker, Aspheric curvatures, refractive indices and chromatic aberration for the rat eye. Vision Res 23, 1351-1363 (1983).
55. D. Schulz, M. E. Iliev, B. E. Frueh, D. Goldblum, In vivo pachymetry in normal eyes of rats, mice and rabbits with the optical low coherence reflectometer. Vision Res 43, 723-728 (2003).
56. N. Washburn, S. Bencherif, A. Srinivasan, synthesis and characterization of highly crosslinked hyaluronan hydrogels. Polymer Preprints 48, (2007).
57. J. J. Lim, J. S. Temenoff, The effect of desulfation of chondroitin sulfate on interactions with positively charged growth factors and upregulation of cartilaginous markers in encapsulated MSCs. Biomaterials 34, 5007-5018 (2013).
58. H. Shirahama, B. H. Lee, L. P. Tan, N. J. Cho, Precise Tuning of Facile One-Pot Gelatin Methacryloyl (GeIMA) Synthesis. Sci Rep 6, 31036 (2016).

What is claimed is:

1. A photosensitive bulking agent (PBA) comprising a photoreactive collagen-like peptide (PCLP) and polyethylene glycol (PEG).

2. The PBA of claim 1, comprising riboflavin.

3. The PBA of claim 1, wherein the PEG is a 2-Arms-PEG acrylate, 4-Arms-PEG acrylate, or an 8-Arms-PEG acrylate.

4. The PBA of claim 1, wherein the PEG is an 8-Arms-PEG acrylate.

5. The PBA of claim 1, wherein the PCLP comprises at least one photoreactive residue.

6. The PBA of claim 5, wherein each of the at least one photoreactive residue is flanked by two aliphatic amino acid residues selected from the group consisting of glycine residues, alanine residues, valine residues and a combination thereof.

7. The PBA of claim 6, wherein the two aliphatic amino acid residues are glycine residues.

8. The PBA of claim 1, wherein the PCLP comprises a plurality of collagen-like folding motifs.

9. The PBA of claim 1, wherein the PCLP comprises at least one cell adhesion motif flanked by the plurality of collagen-like folding motifs.

10. The PBA of claim 9, wherein the at least one cell adhesion motif comprises the amino acid residues GFOGER (SEQ ID NO:1).

11. The PBA of claim 1, which is sensitive to visible light.

12. The PBA of claim 11, wherein the visible light has a wavelength of about 400 nm to about 490 nm.

13. The PBA of claim 11, wherein the visible light is blue light.

14. The PBA of claim 13, wherein the blue light has a wavelength of about 460 nm.

15. The PBA of claim 1, wherein the PCLP comprises the amino acid sequence defined by (X₀)ₘX₁(X₆)ₙ(X₂)ₒ(X₇)ₚX₅(X₈)_{q}; wherein
each of X₀, X₆, X₇, and X₈ independently represent a glycine, an alanine or a valine, wherein 1≤m≤6, 1≤n≤6, 1≤q≤6, and 0≤p≤6;
X₁ and X₅ are photoreactive residues which are the same or different; and
X₂ represent collagen-like folding motifs, wherein 3≤o≤20.

16. The PBA of claim 15, wherein each of X₁ and X₅ represent a lysine, cysteine, tryptophan, tyrosine, or histidine protected by a N-allyloxycarbonyl (Alloc) group, a 9-fluorenylmethoxycarbonyl (Fmoc) group, or a tert-butyloxycarbonyl (Boc) group.

17. The PBA of claim 16, wherein each of X₁ and X₅ represent a lysine protected by a N-allyloxycarbonyl (Alloc) group.

18. The PBA of claim 15, wherein X₂ is defined by amino acid sequence GPO, POG, RGD, GROGER (SEQ ID NO:2), GLOGEN (SEQ ID NO:3), or GMOGER (SEQ ID NO:4).

19. The PBA of claim 18, wherein X₂ is defined by amino acid sequence POG.

20. The PBA of claim 15, wherein o=4.

21. The PBA of claim 15, wherein each of X₀, X₆, X₇, and X₈ represents a glycine.

22. The PBA of claim 15, wherein each of m, n, and q=1 and p=0.

23. The PBA of claim 15, wherein the PCLP comprises the amino acid sequence defined by GK(Alloc)GPOGPOGPOGPOGPOGPOGPOGPOGK(Alloc)G (SEQ ID NO:5), wherein amino acid residue K(Alloc) represents a lysine protected by a N-allyloxycarbonyl (Alloc) group.

24. The PBA of claim 1, wherein the photoreactive collagen-like peptide (PCLP) comprises the amino acid sequence defined by (X₀)ₘX₁(X₆)ₙ(X₂)ₒX₃(X₄)ₚ(X₇)_{g}X₅(X₈)ᵣ; wherein
each of X₀, X₆, X₇, and X₈ independently represent a glycine, an alanine or a valine, wherein 1≤m≤6, 1≤n≤6, 1≤q≤6, and 1≤r≤6;
X₁ and X₅ are photoreactive residues which are the same or different;
X₂ and X₄ are collagen-like folding motifs, wherein X₂ and X₄ are the same or different and 2≤o≤10 and 2≤p≤10; and
X₃ represent at least one cell adhesion motif.

25. The PBA of claim 24, wherein each of X₁ and X₅ represent a lysine, cysteine, tryptophan, tyrosine, or histidine protected by a N-allyloxycarbonyl (Alloc) group, a 9-fluorenylmethoxycarbonyl (Fmoc) group, or a tert-butyloxycarbonyl (Boc) group.

26. The PBA of claim 25, wherein each of X₁ and X₅ represent a lysine protected by a N-allyloxycarbonyl (Alloc) group.

27. The PBA of claim 24, wherein each of X₂ and X₄ is defined by amino acid sequence GPO, POG, RGD, GROGER (SEQ ID NO:2), GLOGEN (SEQ ID NO:3), or GMOGER (SEQ ID NO:4).

28. The PBA of claim 27, wherein each of X₂ and X₄ is defined by amino acid sequence GPO.

29. The PBA of claim 24, wherein each of o and p=4.

30. The PBA of claim 24, wherein each of X₀, X₆, X₇, and X₈ represent a glycine.

31. The PBA of claim 24, wherein each of m, n, q and r=1.

32. The PBA of claim 24, wherein the PCLP comprises the amino acid sequence defined by GK(Alloc)GGPOGPOGPOGPOGFOGERGPOGPOGPOGPOGK(Alloc)G (SEQ ID NO:6), wherein amino acid residue K(Alloc) represents a lysine protected by a N-allyloxycarbonyl (Alloc) group.

33. The PBA of any one of claims 1-32, which is a photosensitive corneal stromal bulking agent.

34. A composition comprising the PBA of any one of claims 1 to 33 and a physiologically acceptable carrier.

35. A scaffold comprising the PBA of any one of claims 1 to 33.

36. The scaffold of claim 35, further comprising hyaluronic acid methacrylate and/or chondroitin methacrylate.

37. A precursor hydrogel obtained by crosslinking a precursor solution comprising the PBA of any one of claims 1 to 33.

38. The precursor hydrogel of claim 37, further comprising hyaluronic acid methacrylate and/or chondroitin methacrylate.

39. The precursor hydrogel of claim 37 or 38, wherein the precursor hydrogel is obtained by crosslinking the precursor solution by irradiating the precursor solution with visible light in vitro.

40. The precursor hydrogel of claim 39, wherein the visible light has a wavelength of about 400 nm to about 490 nm.

41. The precursor hydrogel of claim 39, wherein the visible light is blue light.

42. The precursor hydrogel of claim 41, wherein the blue light has a wavelength of about 460 nm.

43. The precursor hydrogel of claim 37, having a viscosity within about 10% of the viscosity of Viscoat®.

44. The precursor hydrogel of claim 37, for use in preparing a hydrogel.

45. The precursor hydrogel of claim 37, for use in intrastromal injection.

46. The precursor hydrogel of claim 37, for use in treating a corneal disease.

47. The precursor hydrogel of claim 37, for use in treating a corneal disease characterized by a thinning cornea.

48. The precursor hydrogel of claim 37, for use in combination with a cornea implant for treating a corneal disease.

49. A hydrogel comprising the precursor gel of claim 37, wherein the hydrogel is obtained by crosslinking the precursor gel in vivo.

50. The hydrogel of claim 49, wherein crosslinking the precursor gel in vivo is performed by irradiating a subject injected with the precursor solution.

51. The hydrogel of claim 49, wherein the hydrogel is obtained by irradiating the precursor gel with visible light.

52. The hydrogel of claim 51, wherein the visible light has a wavelength of about 400 nm to about 490 nm.

53. The hydrogel of claim 51, wherein the visible light is blue light.

54. The hydrogel of claim 53, wherein the blue light has a wavelength of about 460 nm.

55. The hydrogel of claim 49, comprising pores having a diameter of between about 10 μm to about 50 μm.

56. The hydrogel of claim 49, wherein the hydrogel has a compression modulus within about 10% of the compression modulus of a mammalian cornea.

57. The hydrogel of claim 49, wherein the hydrogel has a shear rate of between about 0 s⁻¹ to about 200 s⁻¹.

58. The precursor hydrogel of claim 37 or the hydrogel of claim 49, wherein the crosslinking allows conjugation of the photoreactive collagen-like peptide (PCLP) with the polyethylene glycol (PEG).

59. A method of preparing a hydrogel in vivo comprising:
providing a precursor solution comprising a photoreactive collagen-like peptide (PCLP), polyethylene glycol (PEG), and riboflavin;
precrosslinking the precursor solution by irradiating the precursor solution with visible light to obtain a precursor hydrogel;
injecting the precursor gel at a desired location in a subject; and
crosslinking the precursor gel by irradiating the desired location in the subject with visible light to obtain a hydrogel in the subject.

60. The method of claim 59, wherein the photoreactive collagen-like peptide (PCLP) comprises at least one photoreactive residue.

61. The method of claim 59, wherein each of the at least one photoreactive residues is flanked by two glycine residues.

62. The method of claim 59, wherein the photoreactive collagen-like peptide (PCLP) comprises a plurality of collagen-like folding residues.

63. The method of claim 59, wherein the photoreactive collagen-like peptide (PCLP) comprises at least one cell adhesion motif flanked by the plurality of collagen-like folding residues.

64. The method of claim 59, wherein the precursor solution comprises hyaluronic acid methacrylate, gelatin methacrylate, chondroitin methacrylate, or any combination thereof.

65. The method of claim 59, wherein the visible light has a wavelength of about 400 nm to about 490 nm.

66. The method of claim 59, wherein the visible light is blue light.

67. The method of claim 65, wherein the blue light comprises a wavelength of about 460 nm.

68. The method of claim 59, wherein the precrosslinking and/or crosslinking are performed under low oxygen concentration.

69. The method of claim 59, wherein the low oxygen concentration is about 1% v/v.

70. The method of claim 59, wherein precrosslinking the precursor solution is performed at 114 mW/cm².

71. The method of claim 59, wherein the precrosslinked precursor solution is centrifuged to remove bubbles therein.

72. The method of claim 59, wherein irradiating the precursor solution and/or irradiating the desired location in the subject is performed by pulsed irradiation.

73. The method of claim 59, wherein the pulsed irradiation comprises irradiating for about 0.5 s to about 5 seconds at intervals of about 0.5 s to about 5 seconds.

74. The method of claim 59, wherein a total light dosage irradiated at the desired location in the subject is about 2.5 J/cm².

75. The method of claim 59, wherein the PCLP comprises at least one cell adhesion motif.

76. The method of claim 59, wherein the hydrogel has a shear rate of between about 0 s⁻¹ to about 200 s⁻¹.

77. The method of claim 59, wherein the photoreactive collagen-like peptide (PCLP) comprises the amino acid sequence defined by (X₀)ₘX₁(X₆)ₙ(X₂)ₒX₃(X₄)ₚ(X₇)_{g}X₅(X₈)ᵣ; wherein
each of X₀, X₆, X₇, and X₈ independently represent a glycine, an alanine or a valine, wherein wherein 1≤m≤6, 1≤n≤6, 1≤q≤6, and 1≤r≤6;
X₁ and X₅ are photoreactive residues which are the same or different;
X₂ and X₄ are collagen-like folding motifs, wherein X₂ and X₄ are the same or different and 2≤o≤10 and 2≤p≤10; and
X₃ represent at least one cell adhesion motif.

78. The method of claim 77, wherein each of X₁ and X₅ represent a lysine, cysteine, tryptophan, tyrosine, or histidine protected by a N-allyloxycarbonyl (Alloc) group, a 9-fluorenylmethoxycarbonyl (Fmoc) group, or a tert-butyloxycarbonyl (Boc) group.

79. The method of claim 77, wherein each of X₁ and X₅ represent a lysine protected by a N-allyloxycarbonyl (Alloc) group.

80. The method of claim 77, wherein each of X₂ and X₄ is defined by amino acid sequence GPO, POG, RGD, GROGER (SEQ ID NO:2), GLOGEN (SEQ ID NO:3), or GMOGER (SEQ ID NO:4).

81. The method of claim 80, wherein each of X₂ and X₄ is defined by amino acid sequence GPO.

82. The method of claim 77, wherein each of o and p=4.

83. The method of claim 77, wherein each of X₀, X₆, X₇, and X₈ represents a glycine.

84. The method of claim 77, wherein each of m, n, q, and r=1.

85. The method of claim 77, wherein the PCLP comprises the amino acid sequence defined by GK(Alloc)GGPOGPOGPOGPOGFOGERGPOGPOGPOGPOGK(Alloc)G (SEQ ID NO:6), wherein amino acid residue K(Alloc) represents a lysine protected by a N-allyloxycarbonyl (Alloc) group.

86. The method of claim 59, wherein the hydrogel has a viscosity within about 10% of the viscosity of Viscoat®.

87. The method of claim 59, wherein the hydrogel has compression modulus within about 10% of the compression modulus of a mammalian cornea.

88. The method of claim 59, wherein crosslinking the precursor gel is performed by irradiating the desired location in the subject with visible light for a period of between about 2 minutes to about 15 minutes.

89. A cornea implant comprising the precursor hydrogel of claim 37 or the hydrogel of claim 49.

90. A method of treating a condition of the eye characterized by a corneal defect, said method comprising:
making a limbal incision in an area affected by the corneal defect in a subject;
administering the precursor hydrogel over the corneal defect; and
irradiating the precursor hydrogel with visible light.

91. A peptide comprising the amino acid sequence GK(Alloc)GPOGPOGPOGPOGPOGPOGPOGPOGK(Alloc)G (SEQ ID NO:5), wherein amino acid residue K(Alloc) represents a lysine protected by a N-allyloxycarbonyl (Alloc) group.

92. A peptide comprising the amino acid sequence GK(Alloc)GGPOGPOGPOGPOGFOGERGPOGPOGPOGPOGK(Alloc)G (SEQ ID NO:6), wherein amino acid residue K(Alloc) represents a lysine protected by a N-allyloxycarbonyl (Alloc) group.

93. A composition comprising the peptide of claim 91 or 92 and an 8-Arms-PEG acrylate.

94. The composition of claim 93, wherein the composition is not crosslinked.

95. The composition of claim 93, wherein the composition is partially crosslinked.

96. The composition of claim 93, wherein the composition is fully crosslinked.

97. Use of the composition of any one of claims 93-96 for treating, preventing, or augmenting corneal thinning or any other corneal defects in a subject in need thereof.
